(19) United States
(12) Patent Application Publication
(10) Pub. No.: US 2026/0297038 A1
(43) Pub. Date: Oct. 1, 2026

(54) ORTHO-PHTHALALDEHYDE-FUNCTIONALIZED ULTRAVIOLETABSORBERS AND VISIBLE CHROMOPHORES FOR SEMI-PERMANENT TOPICAL APPLICATIONS

(71) Applicants: Carson J. BRUNS, Boulder, CO (US); Jesse BUTTERFIELD, Boulder, CO (US); Hyejin KWON, Superior, CO (US); Emily GARDNER, Boulder, CO (US); The Regents of the University of Colorado, a body corporate, Denver, CO (US); HYPRSKN INC., New York, NY (US)

(72) Inventors: Carson J Bruns, Boulder, CO (US); Jesse Butterfield, Boulder, CO (US); Hyejin Kwon, Superior, CO (US); Emily Gardner, Boulder, CO (US)

(73) Assignee: The Regents of the University of Colorado, a body corporate, Denver, CO (US)

(21) Appl. No.: 19/476,828

(22) PCT Filed: Apr. 22, 2024
(86) PCT No.: PCT/US2024/025644

§ 371 Date: Oct. 20, 2025

Related U.S. Application Data
(60) Provisional application No. 63/497,654, filed on Apr. 21, 2023.

(51) Int. Cl. C07D 249/20; A61K 8/35; A61K 8/49; A61Q 17/04; C07C 45/00; C07C 49/215; C07C 49/223; C07D 251/24

(52) U.S. Cl. CPC C07D 249/20; A61K 8/35; A61K 8/496; A61K 8/4966; A61Q 17/04; C07C 45/002; C07C 49/215; C07C 49/223; C07D 251/24

(57) ABSTRACT

Ortho-phthaldehyde is introduced as a versatile moiety to imbue ultraviolet-absorbing or visible chromophore compounds bearing this moiety with the capability to remain locally retained in the epidermis for days to weeks by virtue of a safe, efficient, and reagent-free reaction that bonds the ortho-phthalaldehyde moiety covalently to endogenous proteins in the stratum corneum of skin tissue by forming a phthalimidine linkage with primary aliphatic amines such as lysine and N-terminal residues, when applied topically on human or other mammalian skin from a suitable liquid, gel, or emulsion containing the ortho-phthalaldehyde compound with cosmetically acceptable alcohols, oils, and modifiers.

### TECHNICAL FIELD

The presently claimed invention relates to the design and methods of use for compounds bearing an amine-reactive functional group, ortho-phthalaldehyde, that bonds cosmetic ultraviolet absorbers or visible chromophores to skin proteins covalently, providing a semi-permanent body mark that is retained in the stratum corneum of the epidermis for a period of time lasting from days to weeks.

### BACKGROUND ART

A wide variety of products available on the market are designed to be applied topically to skin for various purposes including medical (such as wound healing ointments or sunscreens), cosmetic (such as moisturizing lotions and makeup), and body art (such as henna and temporary tattoos). Most topical skin products have a relatively short service life of hours to days because they wear off the skin's surface with moisture, abrasion, and natural shedding of superficial skin cells. In many cases, it is desirable to extend the service life of these topical products into multi-day or week timescales. Natural products such as henna and genipin can produce stains on the skin that exhibit these longer service lifetimes because these colorant molecules contain reactive functional groups that bond them covalently to proteins or other biomolecules in the stratum corneum layer of skin. The use of genipin to render semi-permanent visible marks on skin is taught in several patents, including U.S. Pat. No. 10,143,641B2, JP2022521485A, CN210121244U, and CN112428737A. Henna-based compositions and methods are taught in U.S. Pat. No. 9,827,049B2, FR286225A1, and BRMU8803132US, for example. Dihydroxyacetone, commonly used in sunless tanning products, as taught in US2008027972A1 and others, also stains the skin by reacting with residual proteins in the stratum corneum. However, henna, genipin, and dihydroxyacetone cause visibly colored stains on the skin, which may be undesirable for some applications, such as sunscreens and body art, wherein a change in color upon reaction could lead to undesired aesthetic outcomes. These limitations motivate the search for a generalizable strategy to create semi-permanent topical ultraviolet-absorbing agents or visible chromophore agents in which the color of the skin can be controlled and, optionally, does not cause a color change upon reacting with endogenous proteins in skin tissue.

The present invention provides ultraviolet-absorbing agents or visible chromophore agents possessing a chemical moiety, ortho-phthalaldehyde, that can form a phthalimidine bond with endogenous proteins in skin tissue by a reagent-free reaction that does not cause significant visible color change to the agent. The presently claimed invention also provides compositions containing ortho-phthalaldehyde ultraviolet-absorbing agents or visible chromophore agents that render a semi-permanent ultraviolet-absorbing mark or visible mark, respectively, on the skin that can last for multiple days, and methods for topical application of these compositions to bond the agents semi-permanently to the skin. As used herein, the term “semi-permanent” indicates that the ultraviolet-absorbing agent or visible chromophore agent bonds irreversibly to the skin cells of the stratum corneum such that the corresponding mark cannot be removed from the skin until the skin cells to which the agent is bonded are shed.

The reaction of ortho-phthalaldehyde with amines to form N-substituted isoindolin-1-ones, also known as phthalimidines, was first taught in J. Thiele, J. Schneider, Liebigs Annalen 1909, 369, 287. The probable mechanism of the reaction between ortho-phthalaldehyde derivatives and primary amines is taught in M. Alajarín, P. Sánchez-Andrada, C. López-Leonardo, Á. Álvarez, Journal of Organic Chemistry 2005, 70, 7617-7623. The reaction of ortho-phthalaldehyde derivatives with primary amines in isolated proteins, such as lysine and N-terminal residues of antibodies, is taught in, for example, WO2020/103891A1 and C. L. Tung, C. T. T., Wong, E. Y. M. Fung, X. Li, Organic Letters 2016, 18, 2600-2603, but the reaction of ortho-phthalaldehyde compounds with endogenous proteins in the stratum corneum of skin is unprecedented in the literature until now.

### SUMMARY OF THE INVENTION

The presently claimed invention provides a generalizable strategy for day-to-week-long local retention of topically applied ultraviolet absorbers or visible chromophores by integrating one or more ortho-phthalaldehyde functional group into their molecular structure. When incorporating one or more ortho-phthalaldehyde functional group into the molecular structure of any organic or metallo-organic ultraviolet absorber or visible chromophore, the resulting compound becomes capable of reacting with primary amines, such as those found on the N-termini and lysine residues abundant in proteins, in cosmetically acceptable compositions that contain the ortho-phthalaldehyde ultraviolet absorber or visible chromophore when applied topically to the surface of skin.

The presently claimed invention provides ortho-phthalaldehyde functionalized compounds suitable for semi-permanent topical applications, including ultraviolet absorbers and visible chromophores.

The presently claimed invention also provides methods for the preparation of ortho-phthalaldehyde functionalized ultraviolet absorber or visible chromophore compounds.

The presently claimed invention also provides compositions in the form of solutions, gels, or emulsions containing ortho-phthalaldehyde ultraviolet-absorbing agents or visible chromophore agents suitable for topical application on human or other mammalian skin.

The presently claimed invention also provides methods of applying solutions, gels, or emulsions of ortho-phthalaldehyde-functionalized compounds topically on skin, supported by manual tools such as brushes, sponges, and markers, thin-film applicators, or aerosol generators, which enable the solution to remain in contact with the skin for a sufficient time for the phthalimidine-forming reagent to bond the agents to endogenous proteins in the stratum corneum.

### BRIEF DESCRIPTION OF THE DRAWINGS

For a greater understanding of the invention, reference should be made to the following detailed description, taken in connection with the accompanying drawings, in which:

FIG. 1 is a graph of the ¹H nuclear magnetic resonance spectrum (300 MHz) of 3-(3,4-diformylphenyl)propyl 3-(3-(2H-benzo[d][1,2,3]triazol-2-yl)-5-(tert-butyl)-4-hydroxyphenyl)propanoate, prepared according to the process described in Example 3, in CDCl₃ at 21° C.

FIG. 2 is a graph of the ¹H nuclear magnetic resonance spectrum (300 MHz) of 3-(3,4-diformylphenyl)propyl 2-(4-(diethylamino)-2-hydroxybenzoyl)benzoate, prepared according to the process described in Example 4, in CDCl₃ at 21° C.

FIG. 3 is a graph of the ¹H nuclear magnetic resonance spectrum (300 MHz) of 3-(3,4-diformylphenyl)propyl 3-(4-methoxyphenyl)acrylate, prepared according to the process described in Example 5, in CDCl₃ at 21° C.

FIG. 4 is a set of graphs of the ultraviolet-visible absorption spectra of solutions of (a) 4-((3-(2H-benzo[d][1,2,3]triazol-2-yl)-2-hydroxy-5-methylbenzyl)oxy)phthalaldehyde, prepared according to the process described in example 3, and 3-(3-(benzotriazole-2-yl)-5-tert-butyl-4-hydroxyphenyl)propanoic acid, (b) 3-(3-(2H-benzo[d][1,2,3]triazol-2-yl)-5-(tert-butyl)-4-hydroxyphenyl)propanoate, prepared according to the process described in Example 4, and 2-(4-diethylamino-2-hydroxybenzoyl)benzoic acid, (c) 3-(3,4-diformylphenyl)propyl 3-(4-methoxyphenyl)acrylate, prepared according to the process described in example 5, and 4-methoxycinnamic acid. The comparison of these 3-(3,4-diformylphenyl)propyl esters and their corresponding ultraviolet-absorbing carboxylic acids demonstrates that the 3-(3,4-diformylphenyl)propyl moiety does not diminish the ultraviolet-absorbing properties nor introduce substantial visible absorption in the ultraviolet-absorbing ortho-phthalaldehyde compounds.

FIG. 5 is a graph of the ¹H nuclear magnetic resonance spectrum (300 MHz) of 4-[3,3,4,4,5,5-hexafluoro-2-[2-methyl-5-(3,4-diformylphenyl)-3-thienyl]-1-cyclopenten-1-yl]-5-methyl-2-phenylthiazole in CDCl₃ at 21° C.

FIG. 6 is a graph of the ¹H nuclear magnetic resonance spectrum (300 MHz) of 2-(6-(6-(2-(2-(3,4-diformylphenoxy)ethoxy)ethoxy)-1-oxoisoindolin-2-yl)-3-iminio-3H-xanthen-9-yl)benzoate generated in situ from 4,4′-(oxybis(2,1-ethanediyloxy))bis(phthalaldehyde) and rhodamine 110, the ¹H nuclear magnetic resonance spectra of which are also plotted for the sake of comparison, in 1:2 D₂O:CD₃CN at 21° C.

FIG. 7 is a set of annotated visible and ultraviolet photographs of excised porcine skin areas exposed to the composition described in Example 8 according to the method of Example 10, labeled o-phthalaldehyde, accompanied by a positive control based on genipin and three negative controls based on a commercially available organic and mineral SPF-30 sunscreens and permanent black marker, taken before the skin was washed, then after one wash, after five washes, and after five washes and bisection.

FIG. 8 is a set of annotated photographs of human skin exposed in vivo to a composition described in Example 9 according to the method described in Example 11, after approximately (a) 24 hours and (b) 48 hours of the topical application to skin, following two soap-and-water scrubs per day and ordinary abrasion from clothing, wherein the photochromic semi-permanent topical tattoo attributable to the ortho-phthalaldehyde-diarylethene chromophore agent of the composition is pictured before and after a 5-second exposure to a 365 nm ultraviolet lamp, validating the multi-day efficacy of the visible photochromic chromophore in the composition as a semi-permanent topical tattoo agent.

### DETAILED DESCRIPTION OF THE EMBODIMENTS

Ultraviolet photoprotectants such as sunscreens, as well as body art products such as temporary tattoos, both involve marking or staining the skin with compounds that remain locally retained for a period of time and, during this period of local retention, influence the distribution of incident light that penetrates into and reflects from the skin. Ultraviolet absorbers in sunscreens reflect and dissipate incident ultraviolet light but not visible light, whereas visible chromophores in body art products absorb and reflect visible light as well as ultraviolet light. There is a large body of academic and patent literature on these two product categories. The present invention provides a generalizable strategy to bond many organic and metallo-organic ultraviolet-absorbing or visible chromophore compounds to the skin semi-permanently, for a period of days to weeks, by incorporating a functional group that bonds molecules irreversibly to skin cells by forming an colorless phthalimidine linkage with primary amines of the endogenous proteins in the stratum corneum.

The present invention relates to the use of novel compounds, processes, compositions, and methods that enable the bonding of organic or metallo-organic ultraviolet absorbers and/or visible chromophores to human or other mammalian skin tissue, enabling a semi-permanent mark on the skin for days to weeks. The use of ortho-phthalaldehyde for marking skin with long-term colorants or UV protectants is unprecedented until now. The present invention provides a comprehensive platform for marking or staining the skin with invisible UV-protective marks or visible body decorations that persist and remain locally retained for days to weeks on account of their reactions that bond them to skin proteins.

In some embodiments, the presently claimed invention provides families of compounds in the form of organic or metallo-organic dyes and ultraviolet absorbers bearing the ortho-phthalaldehyde functional group.

In some embodiments, the presently claimed invention provides processes for the preparation of ultraviolet absorbers and visible chromophores possessing the ortho-phthalaldehyde functional group.

In some embodiments, the presently claimed invention provides compositions containing ortho-phthalaldehyde-functionalized ultraviolet absorbers or visible chromophore compounds suitable for topical application to skin.

In some embodiments, the presently claimed invention provides devices and methods of applying these ortho-phthalaldehyde-derived compounds topically to human or other mammalian skin.

### Ultraviolet-Absorbing or Visible Chromophore Ortho-Phthalaldehyde Compounds and Their Preparation

The presently claimed invention provides ultraviolet absorbers and visible chromophore compounds bearing ortho-phthalaldehyde functional groups that react with primary amines including those found in proteins in skin to form phthalimidine linkages, processes for preparing such compounds, compositions containing these compositions, and methods and devices for applying these compositions topically to skin.

In some embodiments, the presently claimed invention is directed at compounds of formula A, selected from the group consisting of formulae A1 or A2,

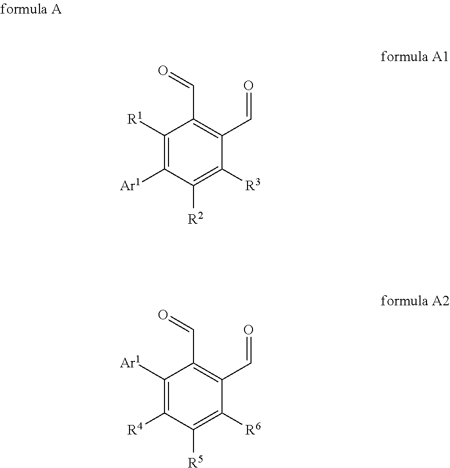

wherein Ar¹ is an ultraviolet absorber moiety of formula B, formula C, formula D, or formula E,

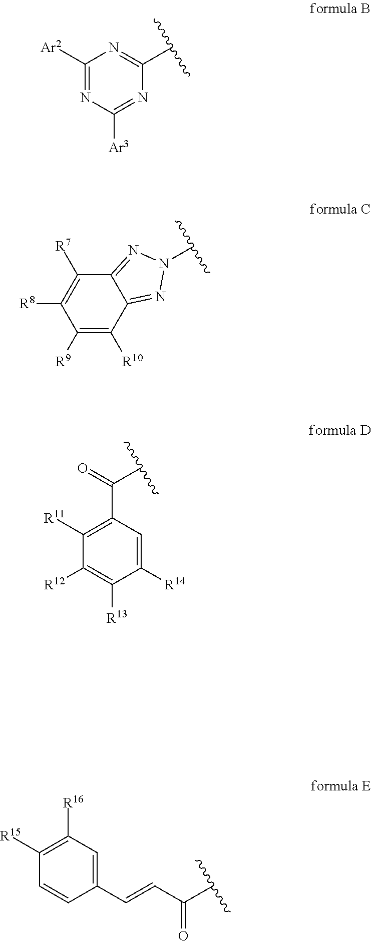

wherein Ar² and Ar³ are independently of each other a moiety of formula F, selected from the group consisting of formulae F1 or F2,

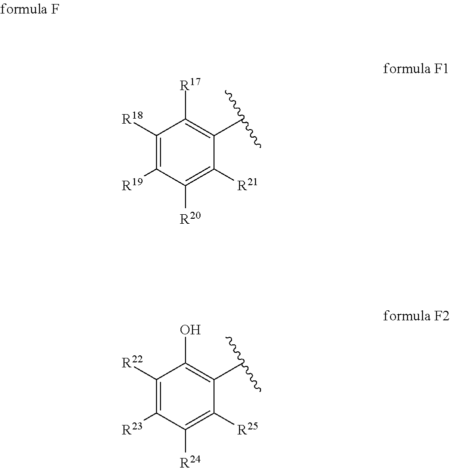

wherein R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, and R²⁵ are independently of each other selected from the group consisting of hydrogen, linear or branched C₁-C₂₄ alkyl, linear or branched C₂-C₂₄ alkenyl, linear or branched C₂-C₂₄ alkynyl, substituted or unsubstituted C₅-C₂₄ cycloalkyl, substituted or unsubstituted C₆-C₂₄ aryl, substituted or unsubstituted C₇-C₂₅ arylalkyl, halogen, CHO, C(═O)R, C(═O)OR, C(═O)OM, CN, OR, NRR′, NO₂, SR, S(═O)₂R, S(═O)₂OR, or S(═O)₂OM, wherein M is an alkali metal, and R and R′ are independently of each other selected from the group consisting of hydrogen, linear or branched C₁-C₂₄ alkyl, substituted or unsubstituted C₆-C₂₄ aryl, substituted or unsubstituted C₇-C₂₅ arylalkyl, or
R² and R³, together with the carbon atoms to which they are bonded, or R⁴ and R⁵, together with the carbon atoms to which they are bonded, or R⁵ and R⁶, together with the carbon atoms to which they are bonded, or R⁷ and R⁸, together with the carbon atoms to which they are bonded, or R⁸ and R⁹, together with the carbon atoms to which they are bonded, or R⁹ and R¹⁰, together with the carbon atoms to which they are bonded, or R¹¹ and R¹², together with the carbon atoms to which they are bonded, or R¹² and R¹³, together with the carbon atoms to which they are bonded, or R¹³ and R¹⁴, together with the carbon atoms to which they are bonded, or R¹⁵ and R¹⁶, together with the carbon atoms to which they are bonded, or R¹⁷ and R¹⁸, together with the carbon atoms to which they are bonded, or R¹⁸ and R¹⁹, together with the carbon atoms to which they are bonded, or R¹⁹ and R²⁰, together with the carbon atoms to which they are bonded, or R²⁰ and R²¹, together with the carbon atoms to which they are bonded, or R²² and R²³, together with the carbon atoms to which they are bonded, or R²³ and R²⁴, together with the carbon atoms to which they are bonded, or R²⁴ and R²⁵, together with the carbon atoms to which they are bonded, form a saturated or unsaturated, substituted or unsubstituted 5- to 19-membered carbocyclic ring that optionally contains 1- to 7-heteroatom substitutions selected from O, N, or S as ring member(s), and wherein at least one of Ar² and Ar³ is a moiety of formula F2, unless R¹, R², or R⁴ is OH, and wherein at least one of R¹, R², or R⁴ is OH when Ar¹ is a moiety of formula C, and wherein at least one of R¹, R², R⁴, or R¹¹ is OH when Ar¹ is a moiety of formula D.

In some embodiments, the presently claimed invention is directed at compounds of formula G, selected from the group consisting of formula G1 or G2,

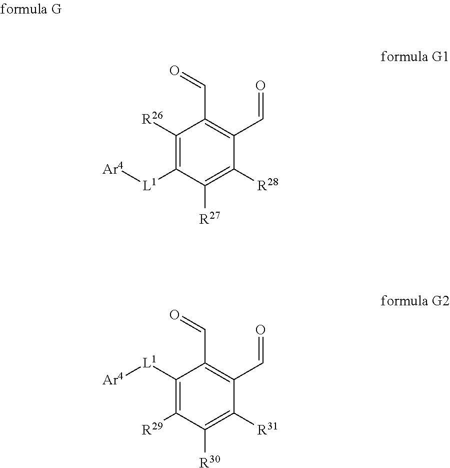

wherein Ar⁴ is a moiety of formula D, or a moiety of formula E, as defined above, or a moiety of formula H, selected from the group consisting of formulae H1, H2, or H3,

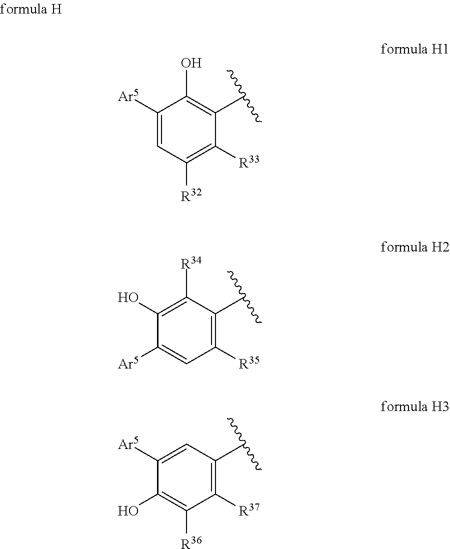

or a moiety of formula I, selected from the group consisting of formulae I1, I2, or I3,

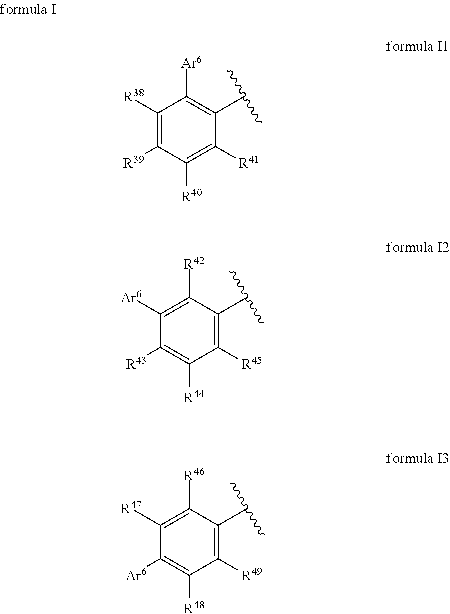

wherein Ar⁵ and Ar⁶ are independently of each other a moiety of formula B, a moiety of formula C, a moiety of formula D, or a moiety of formula E, as defined above, and
L¹ is an optional substituted or unsubstituted linking unit comprising a moiety of formula J, selected from the group consisting of formulae J1, J2, J3, J4, J5, J6, J7, J8, J9, J10, J11, J12, J13, J14, J15, or J16,

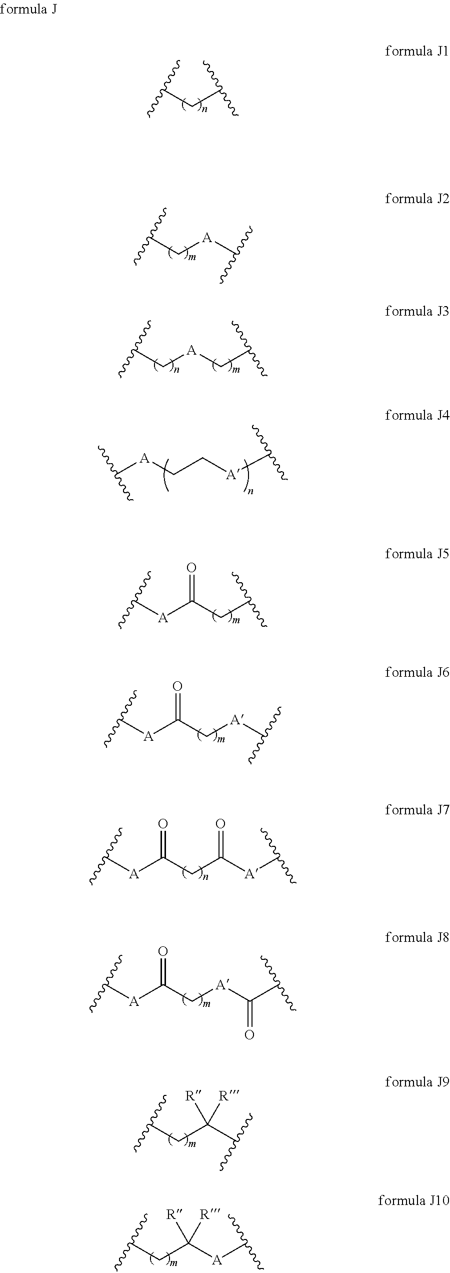

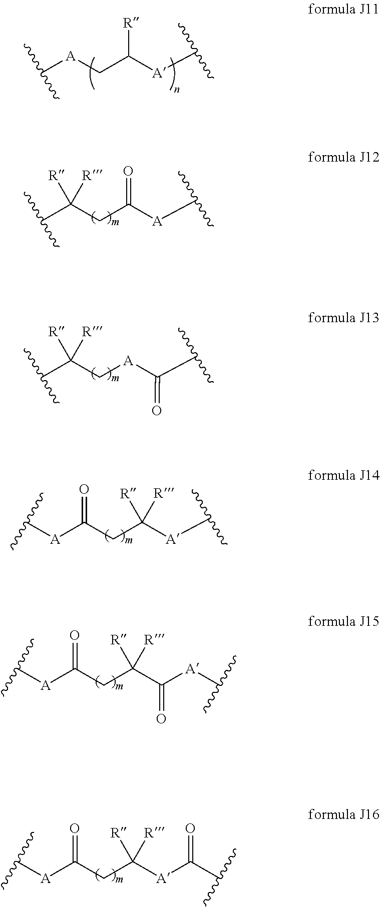

or an optional 1,2-, 1,3-, or 1,4-substituted phenylene linking unit comprising a moiety of formula K, selected from the group consisting of formulae K1, K2, K3, K4, K5, K6, K7, K8, K9, K10, K11, K12, K13, K14, or K15,

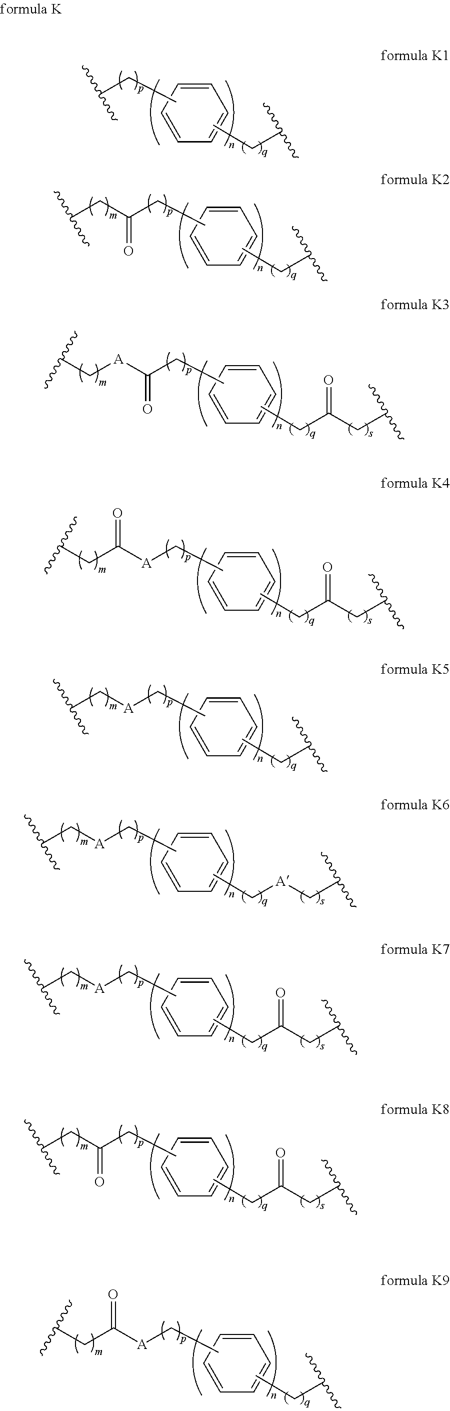

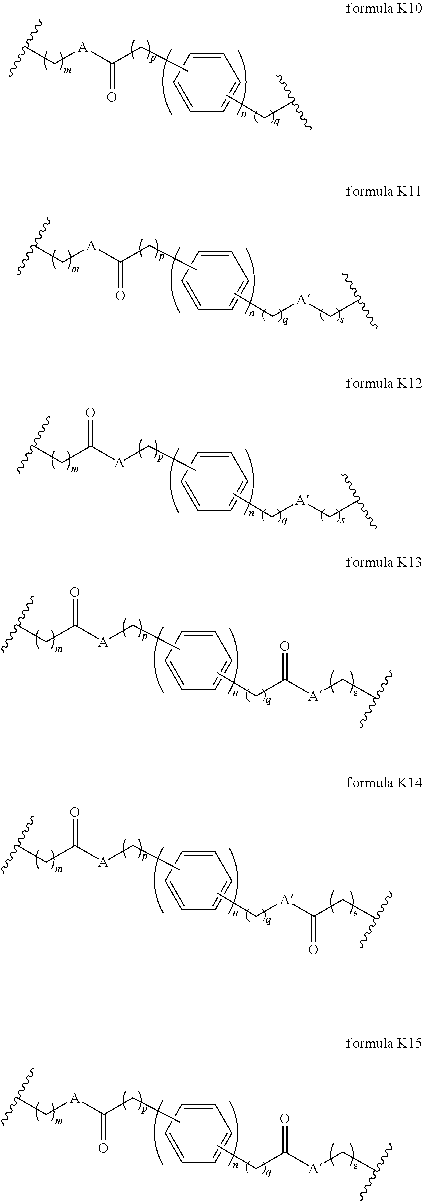

wherein A and A′ are, independently of each other, linking units selected from the group consisting of O, NH, S, SO₂, O(C═O), NH(C═O), NH(C═S), or S(C═O), R″ and R′″ are independently of each other selected from the group consisting of hydrogen, linear or branched C₁-C₂₄ alkyl, substituted or unsubstituted C₆-C₂₄ aryl, substituted or unsubstituted C₇-C₂₅ arylalkyl, C(═O)R, C(═O)OR, or OR, wherein R is selected from the group consisting of hydrogen, linear or branched C₁-C₂₄ alkyl, substituted or unsubstituted C₆-C₂₄ aryl, substituted or unsubstituted C₇-C₂₅ arylalkyl, n denotes a repeating unit that may number from 1 to 24, m, p, q, and s independently of each other denote repeating units that may number from 0 to 24, which may be either monodisperse or polydisperse, and wherein the O(C═O), NH(C═O), NH(C═S), or S(C═O) moieties of A and A′, or the unsymmetrical linking units of formula J or formula K may be oriented in either of the two possible directions, and wherein at least one of Ar² and Ar³ is a moiety of formula F2 when Ar⁴ is a moiety of formula I, unless R³⁸, R⁴², R⁴³, R⁴⁷, or R⁴⁸ is OH, and wherein at least one of R³⁸, R⁴², R⁴³, R⁴⁷, and R⁴⁸ is OH when Ar⁶ is a moiety of formula C, and wherein at least one of R¹¹, R³⁸, R⁴², R⁴³, R⁴⁷, and R⁴⁸ is OH when Ar⁶ is a moiety of formula D.

In some embodiments, the presently claimed invention is directed at processes for the preparation of compounds of formula A or formula G.

In some embodiments, the preparation of compounds of formula A consists of at least the step of reacting at least one compound of formula L, selected from the group consisting of formulae L1, L2, L3, or L4,

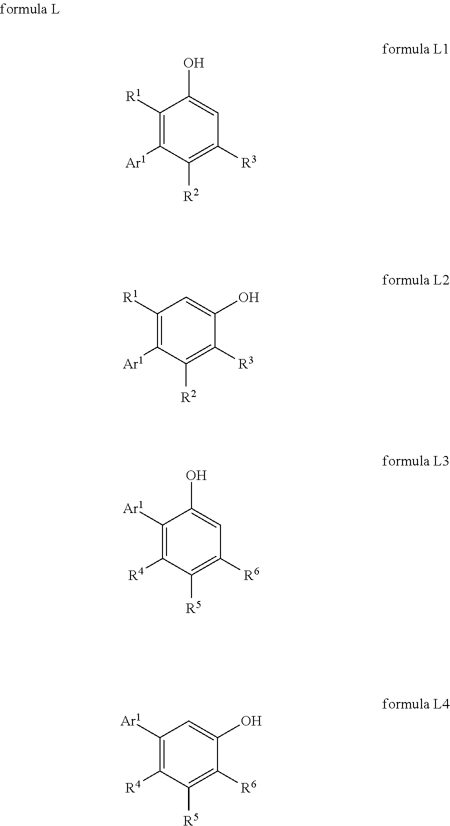

wherein Ar¹, R¹, R², R³, R⁴, R⁵, and R⁶ are as defined above, with any reagents suitable for ortho-formylation of phenols, such as, in some embodiments, paraformaldehyde in the presence of MgCl₂ and NEt₃ (as taught in G. Casnati, M. Crisafulli and A. Ricca, Tetrahedron Letters, 1965, 6, 243-245; G. Casiraghi, G. Casnati, G. Puglia, G. Sartori and G. Terenghi, J. Chem. Soc., Perkin Trans. 1, 1980, 1862; N. U. Hofsløkken and L. Skattebøl, Acta Chem. Scand., 1999, 53, 258-262), or HCN in the presence of a lewis acid such as AlCl₃ (the Gatterman reaction, as taught in L. Gatterman, W. Berchelmann, Berichte der Deutschen Chemischen Gesellschaft 1898, 31, 1765-1769), or chloroform in the presence of a strong base such as the hydroxide anion (the Reimer-Tiemann reaction, as taught in K. Reimer, F. Tiemann, Berichte der Deutschen Chemischen Gesellschaft 1876, 9, 824-828), or a substituted formamide such as N,N-dimethylformamide in the presence of phosphorus oxychloride (the Vilsmeier-Haack reaction, as taught in A. Vilsmeier and A. Haack, Berichte der Deutschen Chemischen Gesellschaft 1927, 60, 119-122; E. Campaigne and W. L. Archer, Journal of the American Chemical Society 1953, 75, 989-991), or hexamine in the presence of a protic acid such as trifluoroacetic acid (the Duff reaction, as taught in J. C. Duff and E. J. Bills, Journal of the Chemical Society 1932, 1987-1988; J. C. Duff, Journal of the Chemical Society 1941, 547-550; J. C. Duff, Journal of the Chemical Society 1945, 276-277), or formamidine acetate in the presence of a dehydrating agent such as acetic anhydride (as taught in S. H. M. Mehr, H. Depmeier, K. Fukuyama, M. Maghami, M. J. MacLachlan, Organic & Biomolecular Chemistry 2017, 15, 581-583), or propanephosphonic acid anhydride, to form salicylaldehyde-derived intermediate compounds of formula M, selected from the group consisting of formulae M1, M2, M3, or M4,

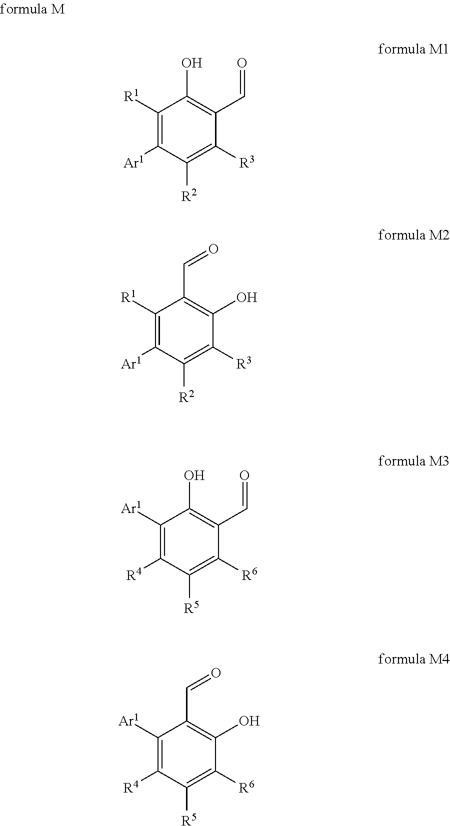

wherein Ar¹, R¹, R², R³, R⁴, R⁵, and R⁶ are as defined above, then reacting the intermediate compound of formula M with formic hydrazide to form a salicylimine-derived compound of formula N, selected from the group consisting of formulae N1, N2, N3, or N4,

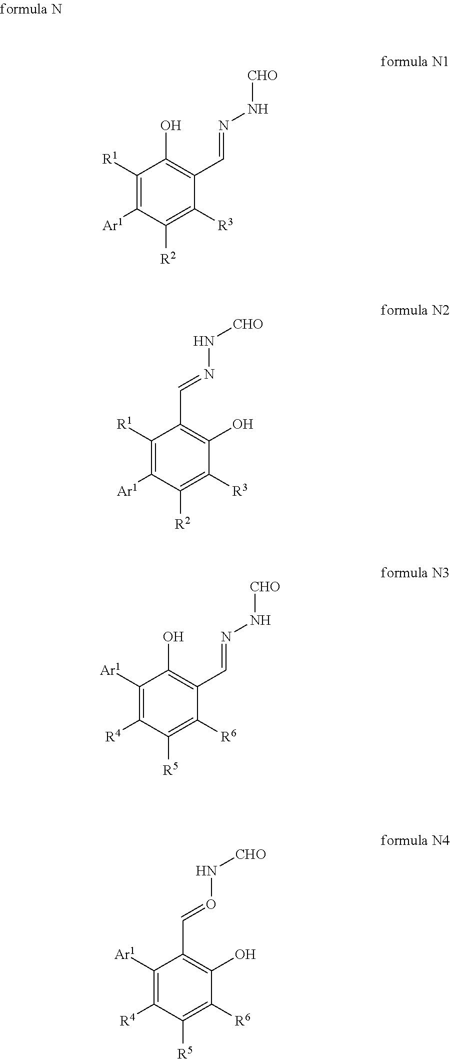

wherein Ar¹, R¹, R², R³, R⁴, R⁵, and R⁶ are as defined above, and reacting the salicylimine derivative intermediate compound of formula N with lead tetraacetate (as taught in A. Kotali, M. Papapetrou, V. Dimos, P. A. Harris, Organic Preparations and Procedures International 1998, 30, 177-181; C. L. Tung, C. T. T. Wong, E. Y. M. Fung, X. Li Organic Letters 2016, 18, 2600-2603) or phenyliodine diacetate or poly(styrene-(iodoso diacetate) (as taught in H. Xian, Z. Qing, Z. Jizheng Synthetic Communications 2001, 31, 2413-2418) to provide the ortho-phthalaldehyde compound of formula A.

### Example 1—Process for the Synthesis of 4-(4,6-bis(2,4-dimethylphenyl)-1,3,5-triazin-2-yl)-5-hydroxyphthalaldehyde

2-(2,4-Dihydroxyphenyl)-4,6-bis(2,4-dimethylphenyl)-1,3,5-triazine (2.0 g, 5.0 mmol), magnesium chloride (1.5 equivalents), paraformaldehyde (6 equivalents), and triethylamine (4 equivalents) is refluxed in acetonitrile for 8 h, then quenched with 5% HCl and extracted into chloroform. The intermediate product 5-(4,6-bis(2,4-dimethylphenyl)-1,3,5-triazin-2-yl)-2,4-dihydroxybenzaldehyde is obtained upon combining and evaporating the volatiles of the organic extracts, and mixing the residue into an ethanolic solution with formic hydrazide (1.1 equivalents) and then refluxed for 2 h. The precipitate of N′-(5-(4,6-bis(2,4-dimethylphenyl)-1,3,5-triazin-2-yl)-2,4-dihydroxybenzylidine) formohydrazide is collected by filtration and dissolved in tetrahydrofuran with lead tetraacetate and stirred at room temperature for 3 h, poured in water, and extracted into dichloromethane three times. The organic extracts are combined, dried over MgSO₄, decanted, concentrated under vacuum, and purified by flash column chromatography on silica gel in 9:1 CHCl₃:EtOAc to afford 4-(4,6-bis(2,4-dimethylphenyl)-1,3,5-triazin-2-yl)-5-hydroxyphthalaldehyde.

In some embodiments, a process for the preparation of compounds of formula A or formula G may comprise at least the step of reacting at least one compound of formula O, selected from the group consisting of formulae O1, O2, O3, or O4,

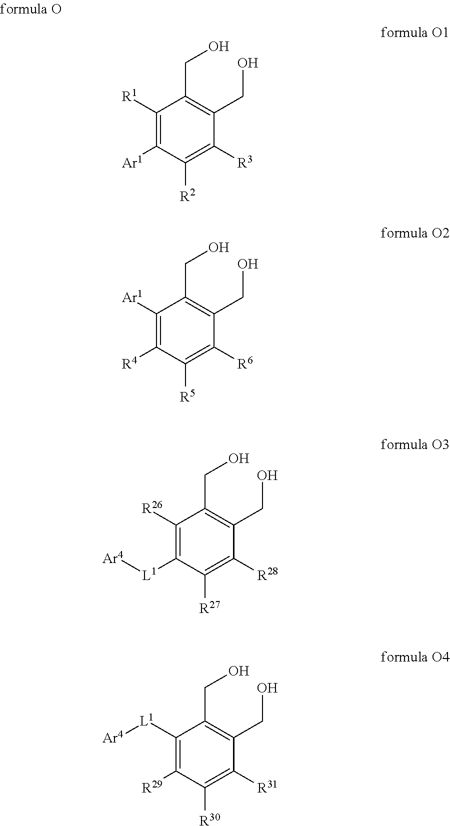

wherein Ar¹, Ar⁴, R¹, R², R³, R⁴, R⁵, R⁶, R²⁶, R²⁷, R²⁸, R²⁹, R³⁰, R³¹, and L¹ are as defined above, with any reagent suitable for oxidizing the ortho-benzylic alcohol moieties of compound of formula O to the corresponding ortho-benzaldehydes, including but not limited to (i) chromium-based reagents such as pyridinium chlorochromate (as taught in E. J. Corey, G. Schmidt, G. Tetrahedron Letters 1979, 5, 399) or pyridinium chromate (as taught in E. J. Corey, J. W. Suggs, Tetrahedron Letters 1975, 2647), or (ii) oxalyl chloride (Swern oxidation, as taught in A. J. Mancuso, S.-L. Huang, D. Swern, Journal of Organic Chemistry 1978, 43, 2480; K. Omura, D. Swern, Tetrahedron 1978, 34, 1651; A. J. Mancuso, D. S. Brownfain, D. Swern, Journal of Organic Chemistry 1979, 44, 4148), or (iii) activated dimethyl sulfoxide reagents mediated by carbodiimides such as 1-ethyl-3-(3-dimethylaminopropyl) carbodiimide, diisopropylcarbodiimide, dicyclohexylcarbodiimide, etc. (Pfitzner-Moffatt oxidation, as taught in K. E. Pfitzner, J. G. Moffatt, Journal of the American Chemical Society 1963, 85, 3027), or mediated by acetic anhydride (Albright-Goldman oxidation, as taught in J. D. Albright, L. Goldman, Journal of the American Chemical Society 1967, 89, 2416), or mediated by phosphorus pentoxide (Albright-Onodera oxidation, as taught in K. Onodera, S. Hirano, N. Kashimura, Journal of the American Chemical Society 1965, 87, 4651), or mediated by sulfur trioxide (Parikh-Doering oxidation, as taught in J. R. Parikh, W. von E. Doering, Journal of the American Chemical Society 1967, 89, 550), or (iv) hypervalent iodine compounds such as Dess-Martin periodinane (as taught in D. B. Dess, J. C. Martin, Journal of Organic Chemistry 1983, 48, 4155), or ortho-iodoxybenzoic acid in dimethyl sulfoxide (IBX, as taught in M. Frigerio, M. Santagostino, Tetrahedron Letters 1994, 35, 8019), or (v) manganese oxide (as taught in S. Ball, T. W. Goodwin, R. A. Morton, Biochemical Journal 1948, 42, 516), (vi) 2,3-dichloro-5,6-dicyano-p-quinone (DDQ, as taught in H.-D. Becker, E. Adler, Acta Chemica Scandinavica 1961, 15, 218; H.-D. Becker, T. Bremholt, Tetrahedron Letters 1973, 197), or (vii) ruthenium-based reagents such as catalytic tetra-n-propylammonium perruthenate (Ley oxidation, as taught in W. P. Griffith, S. V. Ley, G. P. Whitcombe, A. D. White, Journal of the Chemical Society, Chemical Communications 1987, 1625), or (viii) nitroxide radicals such as (2,2,6,6,-tetramethylpiperidin-1-yl)oxanyl (TEMPO) with NaOCl (Anelli oxidation, as taught in P. L. Anelli, C. Biffi, F. Montanari, S. Quici, Journal of Organic Chemistry 1987, 52, 2559) to provide the ortho-phthalaldehyde compound of formula A or formula G.

The synthesis of the ortho-benzylic alcohol compound of formula O1 or O2 may be achieved by a variety of synthetic methods, including but not limited to, by way of example, at least the step of reacting a compound of formula Ar¹X with a compound of formula YAr⁷, or a compound of formula Ar¹Y with a compound of formula XAr⁷, in the presence of a suitable cross-coupling catalyst of palladium(0), palladium(II), palladium (IV), nickel(0), nickel(II), or nickel (IV), wherein X is a leaving group selected from the group consisting of Cl, Br, I, OTs, OTf, OMs, or OH, and Y is selected from the group consisting of SnR₃, wherein R is selected from the group consisting of hydrogen, linear or branched C₁-C₂₄ alkyl, substituted or unsubstituted C₆-C₂₄ aryl, substituted or unsubstituted C₇-C₂₅ arylalkyl (the Migita-Kosugi-Stille reaction, as taught in M. Kosugi, K. Sasazawa, Y. Shikizu, T. Migita, Chemistry Letters, 1977, 6, 301-302; D. Milstein, J. K. Stille, Journal of the American Chemical Society 1978, 100, 3636-3638), ZnX′, wherein X′ is selected from the group consisting of Cl, Br, or I (the Negishi reaction, as taught in A. O. King, N. Okukado, E. Negishi, Journal of the Chemical Society, Chemical Communications 1977, 19, 683; E. Negishi, Accounts of Chemical Research 1982, 15, 340-348), or BXₙ, wherein BXₙ is selected from the group consisting of (i) boronic acid, (ii) an organoborane, including but not limited to 9-borabicyclo[3.3.1]nonane, disiamylborane, diisoproylborane, or dicyclohexylborane, (iii) a boronic ester, including but not limited to those derived from pinacol, neopentyl glycol, 2,3-butanediol, ethylene glycol, 1,3-propanediol, 2-methyl-1,3-propanediol, 2,4-pentanediol, 2,4-dimethyl-2,4-pentanediol, 2,2-diethyl-1,3-propanediol or catechol, (iv) a boronate, including but not limited to sodium trihydroxyboronate, lithium triisopropylboronate, or cyclic triol boronates, (v) N-coordinated boronate, including but not limited to those derived from diethanolamine, N-methyldiethanolamine, N-phenyldiethanolamine, or N-methyliminodiacetic acid, or (vi) triflic borates such as potassium trifluoroborate (the Suzuki-Miyaura reaction, as taught in N. Miyaura, A. Suzuki, Chemical Reviews 1995, 95, 2457-2483), wherein Ar¹ is as defined above, Ar⁷ is a moiety of formula P, selected from the group consisting of formulae P1, P2, P3, or P4,

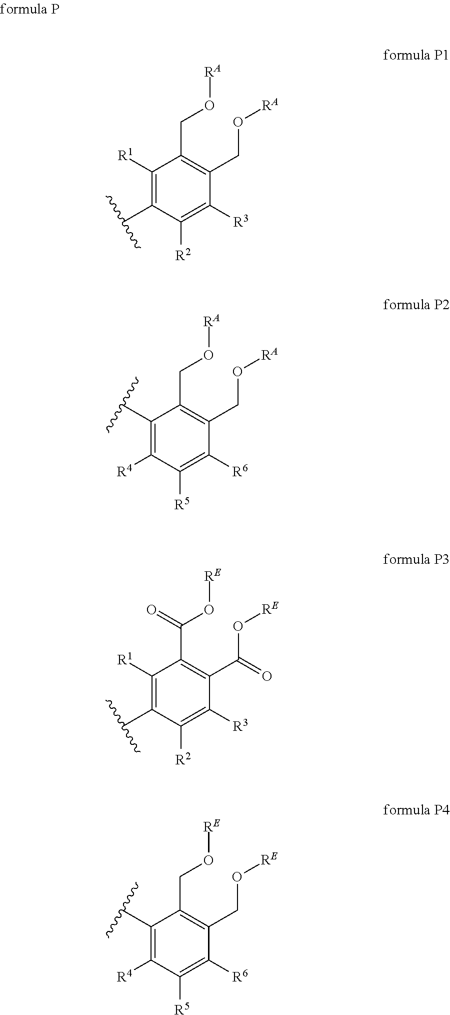

wherein R²⁶, R²⁷, R²⁸, R²⁹, R³⁰, and R³¹ are as defined above and R^{A} is hydrogen or a protecting group for primary alcohols including but not limited those selected from the group consisting of methyl, allyl, methoxymethyl, benzyloxymethyl, 2-(trimethylsilyl)ethoxymethyl, trimethylsilyl, triethylsilyl, triisopropylsilyl, tert-butyldimethylsilyl, Ac, or Bz, and R^{E} is selected from the group consisting of hydrogen, linear or branched C₁-C₂₄ alkyl, or substituted or unsubstituted C₇-C₂₅ arylalkyl, followed by, when R^{A} is not hydrogen, deprotection of the moiety of formula P1 or P2 by at least the step of reacting it with AlCl₃ and Bu₄NI when R^{A} is methyl, or with ^{t}BuLi when R^{A} is allyl, or with trifluoroacetic acid, or HCl, or AcOH, or BF₃·Et₂O with PhSH or 1,3-dithiane, or AlH₂Cl, or AlHCl₂, or BH₃, or LiBF₄, or Ac₂O/FeCl₃ when R^{A} is methoxymethyl, benzyloxymethyl, or 2-(trimethylsilyl)ethoxymethyl, or with K₂CO₃ in MeOH when R^{A} is trimethylsilyl, or with NH₄F, or NBu₄F, or KF/18-crown-6, or trifluoroacetic acid, or AcOH, or TsOH, or HCl when R^{A} is trimethylsilyl, triethylsilyl, triisopropylsilyl, or tert-butyldimethylsilyl, or with K₂CO₃, or KCN, or NaOH, or guanidine, or NH₃, or H₂NNH₂, or 1,8-Diazabicyclo[5.4.0]undec-7-ene, or I₂, or Mg, or Mg(OMe)₂ when R^{A} is Ac or Bz, or by reduction of the carboxylic acid or ester of the moiety of the formula P3 or P4 by at least the step of reacting it with lithium aluminum hydride, diisobutylaluminum hydride, lithium borohydride, sodium borohydride, or borane-dimethylsulfide, to afford the compound of formula O1 or O2.

The synthesis of ortho-benzylic alcohol compounds of formula O3 or O4 may be achieved by a variety of synthetic methods, including but not limited to, in some embodiments, at least the steps of reacting a compound of formula Ar⁴X″ with a compound of formula NuL¹Ar⁸, or a compound of formula Ar⁴L¹X″ with a compound of formula NuAr⁸, or a compound of formula Ar⁴Nu with a compound of formula X″L¹Ar⁸, or a compound of formula Ar⁴L¹Nu with a compound of formula X″ Ar⁸, wherein Ar⁴ and L¹ are as defined above, X″ is a leaving group selected from the group consisting of Cl, Br, I, OTs, OTf, OMs, or OH, or a reactive electrophile selected from the group consisting of an isocyanate, thioisocyanate, epoxide, acyl halide, vinyl sulfone, sulfonyl chloride, aldehyde, ketone, imine, carboxylic acid, ester, anhydride, imide, alkene, alkyne, nitrile, azide, or acyl imidazolide, Nu is a nucleophilic group selected from the group consisting of O⁻, NR⁻, or S⁻ generated in situ by deprotonation with a base, NRR′, or organometallic CM′X generated by metal-halogen exchange, wherein M′ is Li, Na, or Mg, R and R′ are independently of each other selected from the group consisting of hydrogen, linear or branched C₁-C₂₄ alkyl, substituted or unsubstituted C₆-C₂₄ aryl, substituted or unsubstituted C₇-C₂₅ arylalkyl, and Ar⁸ is a moiety of formula Q, selected from the group consisting of formulae Q1, Q2, Q3, or Q4,

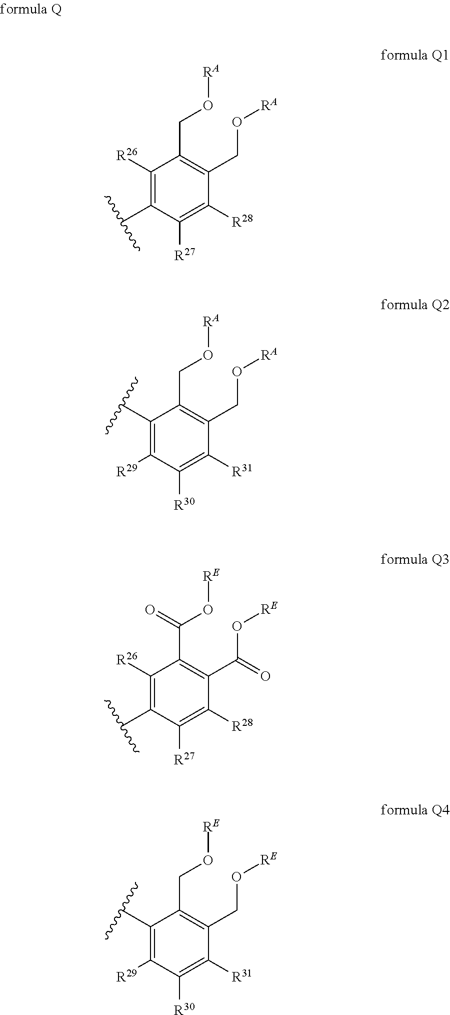

wherein R²⁶, R²⁷, R²⁸, R²⁹, R³⁰, and R³¹ are as defined above and R^{A} is hydrogen or a protecting group for primary alcohols, including but not limited those selected from the group consisting of methyl, allyl, methoxymethyl, benzyloxymethyl, 2-(trimethylsilyl)ethoxymethyl, trimethylsilyl, triethylsilyl, triisopropylsilyl, tert-butyldimethylsilyl, Ac, Bz, and R^{E} is selected from the group consisting of hydrogen, linear or branched C₁-C₂₄ alkyl, substituted or unsubstituted C₇-C₂₅ arylalkyl, such that Nu reacts with X″ to form a covalent bond between Ar⁴ and L¹ or between L¹ and Ar⁸ by, for example, (i) nucleophilic substitution when X″ is Cl, Br, I, OTs, OTf, OMs, or (ii) nucleophilic addition when X″ is isocyanate, thioisocyanate, epoxide, acyl halide, sulfonyl chloride, aldehyde, ketone, imine, carboxylic acid, ester, anhydride, imide, alkene, alkyne, nitrile, azide, or acyl imidazolide, or (iii) esterification, amidation, or thioesterification in the presence of dehydrating agents, including but not limited to carbodiimides such N,N′-dicyclohexylcarbodiimide, N,N′-diisopropylcarbodiimide, 1-ethyl-3-(3-dimethylaminopropyl) carbodiimide, or anhydrides such as acetic anhydride or propanephosphonic acid anhydride, when X″ is the OH terminus of a carboxylic acid moiety, or (iv) triphenylphosphine and diethyl azidodicarboxylate or triphenylphosphine and diisopropyl azidodicarboxylate when X″ is OH and Nu is a carboxylic or phenolic oxygen (the Mitsunobu reaction, as taught in O. Mitsunobu, Y. Yamada, Bulletin of the Chemical Society of Japan 1967, 40, 2380-2382; O. Mitsunobu, Synthesis 1981, 1-28), followed by, when R^{A} is not hydrogen, deprotection of the moiety of formula Q1 or Q2 by at least the step of reacting the compound with AlCl₃ and Bu₄NI when R^{A} is methyl, or with ^{t}BuLi when R^{A} is allyl, or with trifluoroacetic acid, or HCl, or AcOH, or BF₃·Et₂O with PhSH or 1,3-dithiane, or AlH₂Cl, or AlHCl₂, or BH₃, or LiBF₄Ac₂O/FeCl₃ when R^{A} is methoxymethyl, benzyloxymethyl, 2-(trimethylsilyl)ethoxymethyl, or with K₂CO₃ in MeOH, or NH₄F, or NBu₄F, or KF/18-crown-6, or trifluoroacetic acid, or AcOH, or TsOH, or HCl when R^{A} is trimethylsilyl, triethylsilyl, triisopropylsilyl, or tert-butyldimethylsilyl, or with K₂CO₃, or KCN, or NaOH, or guanidine, or NH₃, or H₂NNH₂, or 1,8-Diazabicyclo[5.4.0]undec-7-ene, or I₂, or Mg, or Mg(OMe)₂ when R^{A} is Ac or Bz, or by reduction of the carboxylic acid or ester of the moiety of the formula Q3 or Q4 by at least the step of reacting it with lithium aluminum hydride, or diisobutylaluminum hydride, or lithium borohydride, or sodium borohydride, or borane-dimethylsulfide, to afford the compound of formula 03 or 04.

### Example 2—Process for the Synthesis of 4-((3-(2H-benzo[d][1,2,3]triazol-2-yl)-2-hydroxy-5-methylbenzyl)oxy)phthalaldehyde

The compound 2-[2-(hydroxy-3-(chloromethyl)-5-methyl]benzotriazole, prepared as taught in WO2020118933 A1, is combined with dimethyl 4-hydroxyphthalate (1.1 molar equivalents) and potassium carbonate (3 molar equivalents) in MeCN and the mixture is refluxed for 16 h. The crude reaction mixture is poured into water, extracted into EtOAc three times, then the organic extracts are combined, dried over MgSO₄, decanted, and concentrated to dryness to afford the intermediate compound 1,2-dimethyl 4-[2-hydroxy-3-(2H-benzotriazol-2-yl)-5-methyl]methoxy]-1,2-benzenedicarboxylate, which is, without further purification, dissolved in dry THF and cooled to 0° C. A 1.0 M solution of diisobutylaluminum hydride in heptane (4.5 molar equivalents) is added dropwise to the solution, which is subsequently stirred for 5 h at 60° C. The reaction mixture is poured into water containing four molar equivalents of NaOH, and extracted with CHCl₃ two times to afford, after concentrating the extracts to dryness under vacuum, the intermediate diol compound (4-((3-(2H-benzo[d][1,2,3]triazol-2-yl)-2-hydroxy-5-methylbenzyl)oxy)-1,2-phenylene)dimethanol, which is optionally purified by flash column chromatography on silica gel in 1:1 hexanes:EtOAc eluant. 5 molar equivalents of dimethylsulfoxide is added dropwise to a solution of 2.5 molar equivalents of oxalyl chloride in anhydrous CH₂Cl₂ at −78° C., then the (4-((3-(2H-benzo[d][1,2,3]triazol-2-yl)-2-hydroxy-5-methylbenzyl)oxy)-1,2-phenylene)dimethanol is dissolved in CH₂Cl₂ (2 ml per mmol of diol) and added to the to the reaction mixture after 10 min. After the reaction is stirred under an atmosphere of N₂ at −78° C. for 1 h, Et₃N (1.4 ml per mmol diol) is added dropwise, and the reaction is allowed to warm gradually to ambient temperature while stirring under N₂, then quenched with H₂O and extracted into CH₂Cl₂ three times. The organic extracts are combined, dried of MgSO₄, decanted, concentrated, and purified by flash column chromatography on silica gel in 1:2 hexanes:EtOAc to afford 4-((3-(2H-benzo[d][1,2,3]triazol-2-yl)-2-hydroxy-5-methylbenzyl)oxy)phthalaldehyde.

In some embodiments, a process for the preparation of a compound of formula G may comprise at least the step of reacting at least one protected ortho-phthalaldehyde compound of formula R, selected from the group consisting of formulae R1, R2, R3, or R4,

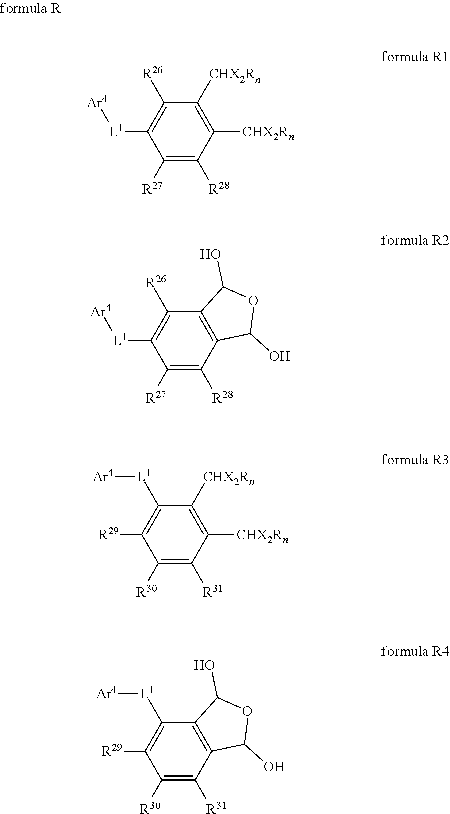

wherein Ar⁴, R²⁶, R²⁷, R²⁸, R²⁹, R³⁰, R³¹, and L¹ are as defined above, CHX₂Rₙ is a protected aldehyde selected from the group consisting of (i) a dialkylacetal, including but not limited to dimethylacetal, diethylacetal, dipropylacetal, dibutylacetal, dipentylacetal, dihexylacetal, diheptylacetal, dioctylacetal, diisopropyl acetal, dicyclohexyl acetal, or dibenzylacetal, or (ii) a cyclic acetal (1,3-dioxanes and 1,3-dioxalanes) of pinacol, neopentyl glycol, 2,3-butanediol, ethylene glycol, 1,3-propanediol, 2-methyl-1,3-propanediol, 2,4-pentanediol, 2,4-dimethyl-2,4-pentanediol, or 2,2-diethyl-1,3-propanediol, or (iii) bis(2-nitrobenzyl)acetal, or (iv) diacetylacetal or other diacylacetals including but not limited to propanoyl, butanoyl, pentanoyl, hexanoyl, heptanoyl, or octanoyl, or decanoyl, or undecanoyl, or dodecanoyl, or (v) a dithioacetal, including but not limited to S,S′-dimethylacetal, S,S′-diethylacetal, S,S′-dipropylacetal, S,S′-dibutylacetal, S,S′-dipentylacetal, S,S′-dihexylacetal, S,S′-diphenylacetal, or S,S′-dibenzylacetal, or (vi) a cyclic dithioacetal, including but not limited to 1,3-dithiane or 1,3-dithiolane, with an acid such as CF₃COOH, or CCl₃COOH, or AcOH, or TsOH, or HCO₂H, or oxalic acid, or HCl, or HBr, or HI, or HNO₃, or H₂SO₄, or H₃PO₄, or perchloric acid, or pyridinium tosylate, or wet MgSO₄, or Amberlyst-15 resin, or water/dimethylsulfoxide at temperatures exceeding 140° C., or thiourea in refluxing EtOH/H₂O, or PPh₃ and CBr₄, or ZnCl₂ with Me₂S and AcCl, or LiBF₄, or CuSO₄·SiO₂, or FeCl₃·SiO₂ when CHX₂Rₙ is dimethylacetal, diethylacetal, dipropylacetal, dibutylacetal, dipentylacetal, dihexylacetal, diheptylacetal, dioctylacetal, diisopropyl acetal, dicyclohexyl acetal, or dibenzylacetal, or with H₂ and Pd/C when CHX₂Rₙ is dibenzylacetal, or by photolysis at 350 nm when CHX₂Rₙ is bis(2-nitrobenzyl)acetal, or with NaOH, or K2CO₃, or alumina, or AlCl₃, or BiCl₃, or potassium 3-dimethylaminophenoxide, or expansive Graphite, or Zeolite Y, or silica gel, or montmorillonite clay, or Fe₂(SO₄)₃, or CBr₄, or SnCl₂, or NaHSO₄ when CHX₂Rₙ is diacetylacetal or another diacylacetal, or with AgNO₃/Ag₂O, or AgClO₄, or FeCl₃, or HgCl₂ and CaCO₃, or SO₂Cl₂ and SiO₂·H₂O, or dimethylsulfoxide at temperatures exceeding 140° C., or I₂, or cetyltrimethylammonium bromide in a halogenated organic solvent, or H₂O₂, or NaIO₄, or CuCl and CuO, or 2,3-dichloro-5,6-dicyano-p-quinone when CHX₂Rₙ is S,S′-dimethylacetal, S,S′-diethylacetal, S,S′-dipropylacetal, S,S′-dibutylacetal, S,S′-dipentylacetal, S,S′-dihexylacetal, S,S′-diphenylacetal, S,S′-dibenzylacetal, 1,3-dithiane, or 1,3-dithiolane, to afford the compound of formula G, wherein the synthesis of a protected ortho-phthalaldehyde compound of formula Q may be achieved by a wide variety of synthetic methods, including but not limited to, by way of example, at least the step of reacting a compound of formula Ar⁴X″ with a compound of formula NuL¹Ar⁹, or a compound of formula Ar⁴L¹X″ with a compound of formula NuAr⁹, or a compound of formula Ar⁴Nu with a compound of formula X″L¹Ar⁹, or a compound of formula Ar⁴L¹Nu with a compound of formula X″Ar⁹, wherein Ar⁴ and L¹ are as defined above, X″ is either a leaving group selected from the group consisting of Cl, Br, I, OTs, OTf, OMs, or OH, or a reactive electrophile selected from the group consisting of an isocyanate, thioisocyanate, epoxide, acyl halide, vinyl sulfone, sulfonyl chloride, aldehyde, ketone, imine, carboxylic acid, ester, anhydride, imide, alkene, alkyne, nitrile, azide, or acyl imidazolide, Nu is a nucleophilic group selected from the group consisting of O⁻, NR⁻, or S⁻ generated in situ by deprotonation with a base, NRR′, or organometallic CM′X generated by metal-halogen exchange, wherein M′ is Li, Na, or Mg, R and R′ are independently of each other selected from the group consisting of hydrogen, linear or branched C₁-C₂₄ alkyl, substituted or unsubstituted C₆-C₂₄ aryl, substituted or unsubstituted C₇-C₂₅ arylalkyl, and Ar⁹ is a moiety of formula S, selected from the group consisting of formulae S1, S2, S3, or S4,

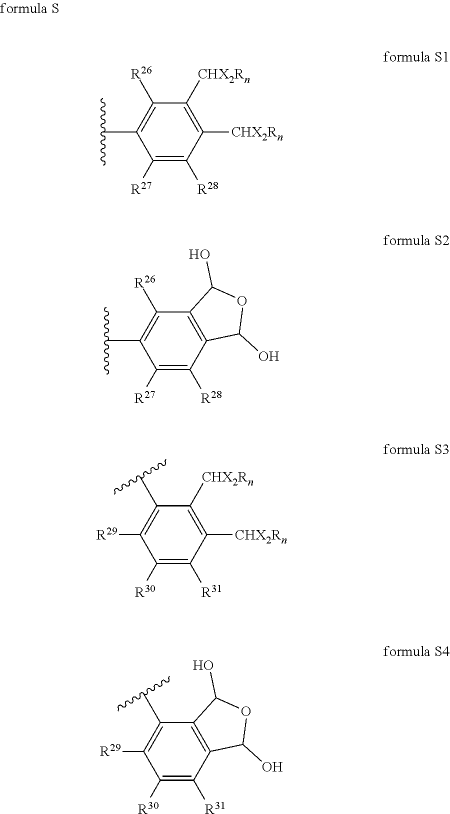

wherein R²⁶, R²⁷, R²⁸, R²⁹, R³⁰, R³¹, and CHX₂Rₙ are as defined above, such that Nu displaces X″ to form a bond between Ar⁴ and L¹, or between L¹ and Ar⁹, by, (i) nucleophilic substitution when X″ is Cl, Br, I, OTs, OTf, or OMs, or (ii) nucleophilic addition when X″ is isocyanate, thioisocyanate, epoxide, acyl halide, sulfonyl chloride, aldehyde, ketone, imine, carboxylic acid, ester, anhydride, imide, alkene, alkyne, nitrile, azide, or acyl imidazolide, or (iii) esterification, amidation, or thioesterification in the presence of dehydrating agents, including but not limited to carbodiimides such N,N′-dicyclohexylcarbodiimide, N,N′-diisopropylcarbodiimide, 1-ethyl-3-(3-dimethylaminopropyl) carbodiimide, or anhydrides such as acetic anhydride or propanephosphonic acid anhydride, when X″ is the OH terminus of a carboxylic acid moiety, or (iv) etherification or esterification in the presence of triphenylphosphine and diethyl azidodicarboxylate or triphenylphosphine and diisopropyl azidodicarboxylate when X″ is OH and Nu is a carboxylic or phenolic oxyanion generated in situ from the corresponding carboxylic acid or phenol moiety (the Mitsunobu reaction, as taught in O. Mitsunobu, Y. Yamada, Bulletin of the Chemical Society of Japan 1967, 40, 2380-2382; O. Mitsunobu, Synthesis 1981, 1-28).

### Example 3—Process for the Synthesis of 3-(3,4-diformylphenyl)propyl 3-(3-(2H-benzo[d][1,2,3]triazol-2-yl)-5-(tert-butyl)-4-hydroxyphenyl)propanoate

0.30 grams (1.1 mmol) of 3-(3,4-di(1,3-dioxolan-2-yl)phenyl)propan-1-ol, prepared as taught in Q. Zhang, Y. Zhang, H. Liu, H. Yee, R. Tian, Y. M. E. Fung, X. Li Biochemistry 2020, 59, 175-178, was combined with 3-(3-(benzotriazole-2-yl)-5-tert-butyl-4-hydroxyphenyl)propanoic acid (0.36 g, 1.1 mmol), N,N′-dicyclohexylcarbodiimide (0.27 g, 1.3 mmol), triethylamine (0.45 g, 4.4 mmol), and catalytic 4-dimethylaminopyridine (7 mg) in CH₂Cl₂ (10 ml) and the reaction mixture was stirred in a sealed roundbottom flask at ambient temperature for 18 h, then washed with water (50 ml) and the aqueous phase was extracted with EtOAc (80 ml) three times. The organic extracts were combined, dried over MgSO₄, decanted, and concentrated to dryness under vacuum. Flash column chromatography on silica gel, eluting with 3:1 hexanes:EtOAc, afforded the intermediate compound 3-(3,4-di(1,3-dioxolan-2-yl)phenyl)propyl 3-(3-(2H-benzo[d][1,2,3]triazol-2-yl)-5-(tert-butyl)-4-hydroxyphenyl)propanoate (0.13 g, 20%). 0.11 grams (0.18 mmol) of 3-(3,4-di(1,3-dioxolan-2-yl)phenyl)propyl 3-(3-(2H-benzo[d][1,2,3]triazol-2-yl)-5-(tert-butyl)-4-hydroxyphenyl)propanoate was dissolved in CH₂Cl₂ (10 ml) and combined with trifluoroacetic acid (0.1 ml) and the reaction was stirred in a covered roundbottom flask at ambient temperature for 16 h. The reaction mixture was washed with water (50 ml) and the aqueous phase was extracted with EtOAc (80 ml) three times. The organic extracts were combined, dried over MgSO₄, decanted, and concentrated to dryness to afford 3-(3,4-diformylphenyl)propyl 3-(3-(2H-benzo[d][1,2,3]triazol-2-yl)-5-(tert-butyl)-4-hydroxyphenyl)propanoate (0.032 g, 34%). To validate the identity of the compound, a graph of the ¹H NMR spectrum of 3-(3,4-diformylphenyl)propyl 3-(3-(2H-benzo[d][1,2,3]triazol-2-yl)-5-(tert-butyl)-4-hydroxyphenyl)propanoate in CDCl₃ is plotted in FIG. 1.

### Example 4—Process for the Synthesis of 3-(3,4-diformylphenyl)propyl 2-(4-(diethylamino)-2-hydroxybenzoyl)benzoate

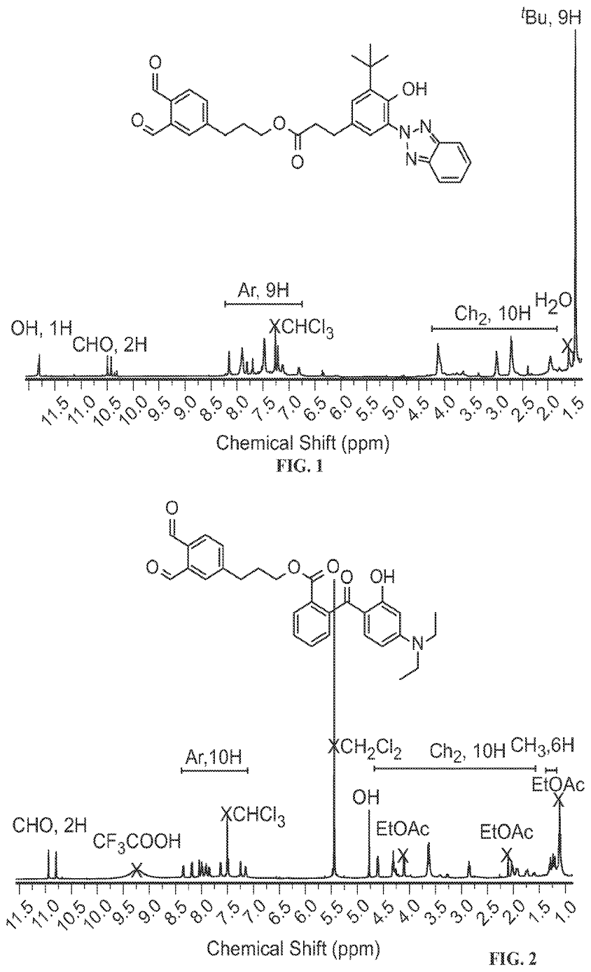

0.30 grams (1.1 mmol) of 3-(3,4-di(1,3-dioxolan-2-yl)phenyl)propan-1-ol, prepared as taught in Q. Zhang, Y. Zhang, H. Liu, H. Yee, R. Tian, Y. M. E. Fung, X. Li Biochemistry 2020, 59, 175-178, was combined with 2-(4-diethylamino-2-hydroxybenzoyl)benzoic acid (0.34 g, 1.1 mmol), N,N′-dicyclohexylcarbodiimide (0.27 g, 1.3 mmol), triethylamine (0.45 g, 4.4 mmol), and catalytic 4-dimethylaminopyridine (7 mg) in CH₂Cl₂ (10 ml) and the reaction mixture was stirred in a sealed roundbottom flask at ambient temperature for 18 h, then washed with water (50 ml) and the aqueous phase was extracted with EtOAc (80 ml) three times. The organic extracts were combined, dried over MgSO₄, decanted, and concentrated to dryness under vacuum. Flash column chromatography on silica gel, eluting with 1:1 hexanes:EtOAc, afforded the intermediate compound 3-(3,4-di(1,3-dioxolan-2-yl)phenyl)propyl 2-(4-(diethylamino)-2-hydroxybenzoyl)benzoate (0.49 g, 80%). 0.49 grams (0.085 mmol) of 3-(3,4-di(1,3-dioxolan-2-yl)phenyl)propyl 2-(4-(diethylamino)-2-hydroxybenzoyl)benzoate was dissolved in CH₂Cl₂ (10 ml) and combined with trifluoroacetic acid (0.1 ml) and the reaction was stirred in a covered roundbottom flask at ambient temperature for 16 h. The reaction mixture was washed with water (50 ml) and the aqueous phase was extracted with EtOAc (80 ml) three times. The organic extracts were combined, dried over MgSO₄, decanted, and concentrated to dryness to afford 3-(3,4-diformylphenyl)propyl 2-(4-(diethylamino)-2-hydroxybenzoyl)benzoate (0.5 g, 100%). To validate the identity of the compound, a graph of the ¹H NMR spectrum of 3-(3,4-diformylphenyl)propyl 2-(4-(diethylamino)-2-hydroxybenzoyl)benzoate in CDCl₃ is plotted in FIG. 2.

### Example 5—Process for the Synthesis of 3-(3,4-diformylphenyl)propyl 3-(4-methoxyphenyl)acrylate

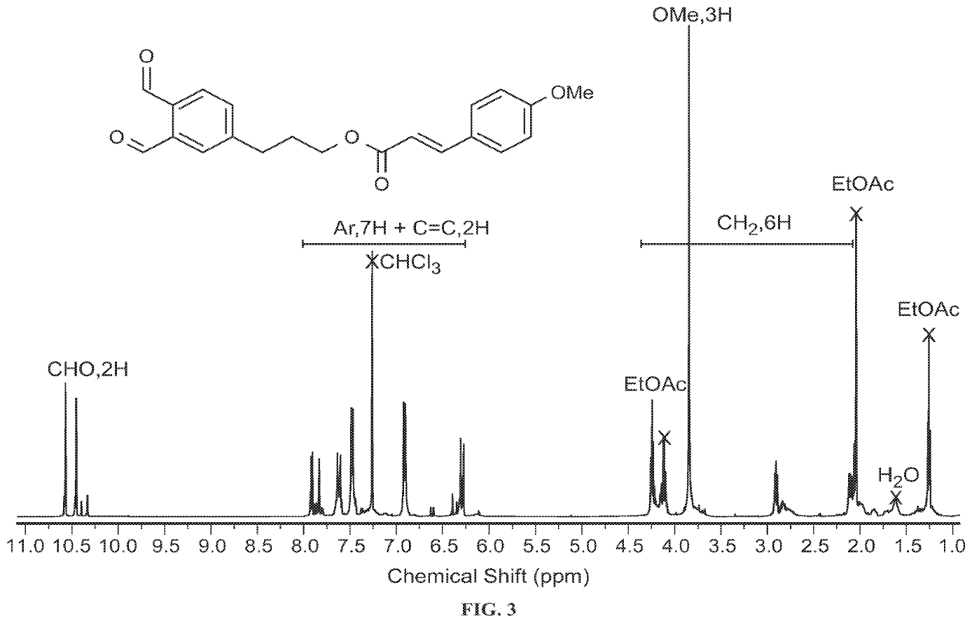

0.30 grams (1.1 mmol) of 3-(3,4-di(1,3-dioxolan-2-yl)phenyl)propan-1-ol, prepared as taught in Q. Zhang, Y. Zhang, H. Liu, H. Yee, R. Tian, Y. M. E. Fung, X. Li Biochemistry 2020, 59, 175-178, was combined with 4-methoxycinnamic acid (0.19 g, 1.1 mmol), N,N′-dicyclohexylcarbodiimide (0.27 g, 1.3 mmol), triethylamine (0.45 g, 4.4 mmol), and catalytic 4-dimethylaminopyridine (7 mg) in CH₂Cl₂ (5 ml) and the reaction mixture was stirred in a sealed roundbottom flask at ambient temperature for 17 h, then washed with water (50 ml) and the aqueous phase was extracted with EtOAc (100 ml) four times. The organic extracts were combined, dried over MgSO₄, decanted, and concentrated to dryness under vacuum. Flash column chromatography on silica gel, eluting with 1:1 hexanes:EtOAc, afforded the intermediate compound 3-(3,5-di(1,3-dioxolan-2-yl)phenyl)propyl 3-(4-methoxyphenyl)acrylate (0.13 g, 28%). 0.13 grams (0.030 mmol) of 3-(3,5-di(1,3-dioxolan-2-yl)phenyl)propyl 3-(4-methoxyphenyl)acrylate was combined with para-toluenesulfonic acid (5 mg, 0.03 mmol) in acetone (5 ml) and the reaction was stirred in a covered roundbottom flask at ambient temperature for 18 h. The reaction mixture was poured in water (50 ml) and the aqueous phase was extracted with EtOAc (50 ml) three times. The organic extracts were combined, dried over MgSO₄, decanted, and concentrated to dryness. Flash column chromatography, eluting with 1:1 hexanes:EtOAc, afforded 3-(3,4-diformylphenyl)propyl 3-(4-methoxyphenyl)acrylate (0.071 g, 68%). To validate the identity of the compound, a graph of ¹H NMR spectrum of 3-(3,4-diformylphenyl)propyl 3-(4-methoxyphenyl)acrylate in CDCl₃ is plotted in FIG. 3.

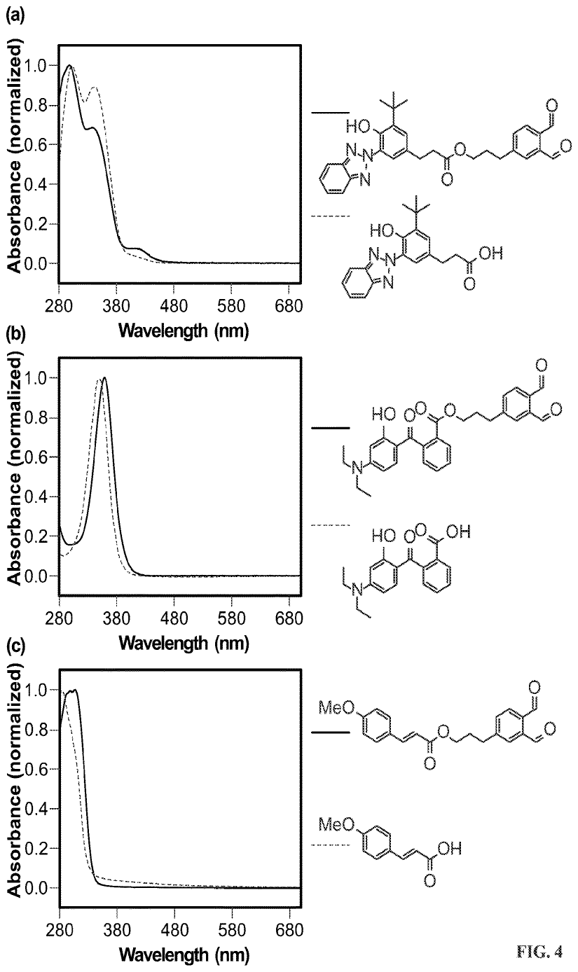

Ultraviolet-visible absorption spectra of the compounds prepared according to the processes described in Example 3, Example 4, and Example 5 are plotted in FIG. 4, demonstrating that the 3-(3,4-diformylphenyl)propyl moiety causes only minor alteration of the absorption profiles of the ultraviolet-absorbing 2-(2-Hydroxyphenyl)-2H-benzotriazole, 2-hydroxybenzophenone, or cinnamate moieties, respectively, without introducing substantial visible absorption, which is favorable for photoprotection of the skin without altering its natural color.

In some embodiments of the presently claimed invention, the ortho-phthalaldehyde moiety employed to react with primary amines to form phthalimidine linkages with endogenous proteins in skin tissue may optionally comprise or be bonded to a visible chromophore, enabling the introduction of a semi-permanent visible mark on the skin when applied in an appropriate topical composition.

In some embodiments, and by way of example, the visible chromophore may be a P-type photochromic moiety capable of being photo-isomerized reversibly between two thermally stable states, at least one of which comprises a visible chromophore moiety, following irradiation with different frequencies of ultraviolet or visible light, constituting a diarylethene—such as those taught in JPH08119963A, JP2002145878A, and others—of formula T, selected from the group consisting of formulae T1 or T2,

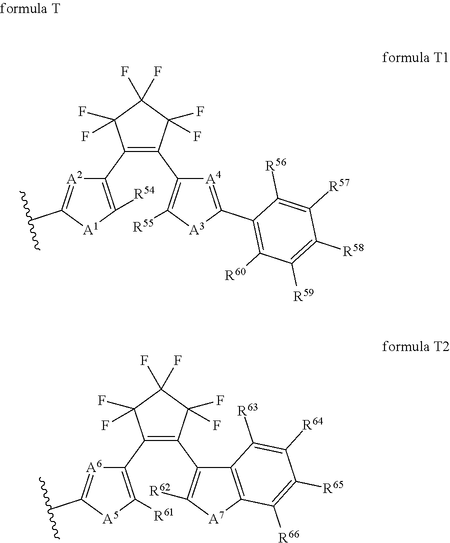

wherein A¹, A³, A⁵, and A⁷ are independently of each other an atom selected from the group consisting of O or S; A², A⁴, and A⁶ are independently of each other selected from the group consisting of N or CR, wherein R is selected from the group consisting of hydrogen, linear or branched C₁-C₂₄ alkyl, substituted or unsubstituted C₆-C₂₄ aryl, substituted or unsubstituted C₇-C₂₅ arylalkyl, R⁵⁴, R⁵⁵, R⁶¹, and R⁶² are independently of each other selected from the group consisting of linear or branched C₁-C₂₄ alkyl, C₇-C₂₅ arylalkyl, or OR^{D}, wherein R^{D} is independently selected from the group consisting of hydrogen, linear or branched C₁-C₂₄ alkyl, substituted or unsubstituted C₇-C₂₅ arylalkyl, R⁵⁶, R⁵⁷, R⁵⁸, R⁵⁹, R⁶⁰, R⁶³, R⁶⁴, R⁶⁵, and R⁶⁶ are independently of each other selected from the group consisting of hydrogen, linear or branched C₁-C₂₄ alkyl, linear or branched C₂-C₂₄ alkenyl, linear or branched C₂-C₂₄ alkynyl, substituted or unsubstituted C₅-C₂₄ cycloalkyl, substituted or unsubstituted C₆-C₂₄ aryl, substituted or unsubstituted C₇-C₂₅ arylalkyl, halogen, C(═O)R, C(═O)OR, C(═O)OM, CN, OR, NRR′, NO₂, SR, S(═O)₂R, S(═O)₂OR, or S(═O)₂OM wherein M is an alkali metal, and R and R′ are independently selected from the group consisting of hydrogen, linear or branched C₁-C₂₄ alkyl, substituted or unsubstituted C₆-C₂₄ aryl, substituted or unsubstituted C₇-C₂₅ arylalkyl, or R⁵⁶ and R⁵⁷, together with the carbon atoms to which they are bonded, or R⁵⁷ and R⁵⁸, together with the carbon atoms to which they are bonded, or R⁵⁸ and R⁵⁹, together with the carbon atoms to which they are bonded, or R⁵⁹ and R⁶⁰, together with the carbon atoms to which they are bonded, or R⁶³ and R⁶⁴, together with the carbon atoms to which they are bonded, or R⁶⁴ and R⁶⁵, together with the carbon atoms to which they are bonded, or R⁶⁵ and R⁶⁶, together with the carbon atoms to which they are bonded, form a saturated or unsaturated, substituted or unsubstituted 5- to 19-membered carbocyclic ring that optionally contains 1- to 7-heteroatom substitutions selected from O, N, or S as ring member(s).

In some embodiments, the presently claimed invention is directed at a process for the preparation of an ortho-phthalaldehyde-functionalized ultraviolet absorber or visible chromophore compound of formula U, selected from the group consisting of formulae U1 or U2,

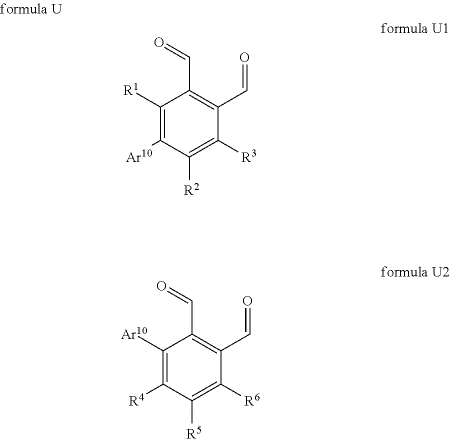

wherein R¹, R², R³, R⁴, R⁴, and R⁶ are as defined above, Ar¹⁰ is either equivalent to Ar¹—an ultraviolet absorbing moiety of formula B, formula C, formula D, or formula E as defined above—or an organic or metallo-organic visible chromophore moiety, or photochromic moiety that reacts to visible light in the ground state or photostationary state, such as a moiety of formula T as defined above, in some embodiments, comprising at least the step of reacting at least one protected ortho-phthalaldehyde compound of formula V, selected from the group consisting of formulae V1, V2, V3, or V4,

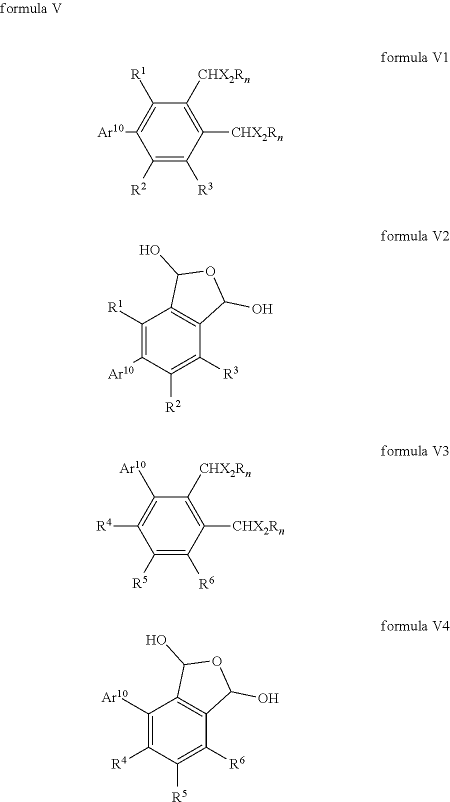

wherein Ar¹⁰, R¹, R², R³, R⁴, R⁵, R⁶, R⁵⁰, R⁵¹, R⁵², R⁵³, and CHX₂Rₙ are as defined above, with an acid such as CF₃COOH, or CCl₃COOH, or AcOH, or TsOH, or HCO₂H, or oxalic acid, or HCl, or HBr, or HI, or HNO₃, or H₂SO₄, or H₃PO₄, or perchloric acid, or pyridinium tosylate, or wet MgSO₄, or Amberlyst-15 resin, or water/dimethylsulfoxide at temperatures exceeding 140° C., or thiourea in refluxing EtOH/H₂O, or PPh₃ and CBr₄, or ZnCl₂ with Me₂S and AcCl, or LiBF₄, or CuSO₄·SiO₂, or FeCl₃·SiO₂ when CHX₂Rₙ is dimethylacetal, diethylacetal, dipropylacetal, dibutylacetal, dipentylacetal, dihexylacetal, diheptylacetal, dioctylacetal, diisopropyl acetal, dicyclohexyl acetal, or dibenzylacetal, or with H₂ and Pd/C when CHX₂Rₙ is dibenzylacetal, or by photolysis at 350 nm when CHX₂Rₙ is bis(2-nitrobenzyl)acetal, or with NaOH, or K₂CO₃, or alumina, or AlCl₃, or BiCl₃, or potassium 3-dimethylaminophenoxide, or expansive Graphite, or Zeolite Y, or silica gel, or montmorillonite clay, or Fe₂(SO₄)₃, or CBr₄, or SnCl₂, or NaHSO₄ when CHX₂Rₙ is diacetylacetal or another diacylacetal, or with AgNO₃/Ag₂O, or AgClO₄, or FeCl₃, or HgCl₂ and CaCO₃, or SO₂Cl₂ and SiO₂·H₂O, or dimethylsulfoxide at temperatures exceeding 140° C., or I₂, or cetyltrimethylammonium bromide in a halogenated organic solvent, or H₂O₂, or NaIO₄, or CuCl and CuO, or 2,3-dichloro-5,6-dicyano-p-quinone when CHX₂Rₙ is S,S′-dimethylacetal, S,S′-diethylacetal, S,S′-dipropylacetal, S,S′-dibutylacetal, S,S′-dipentylacetal, S,S′-dihexylacetal, S,S′-diphenylacetal, S,S′-dibenzylacetal, 1,3-dithiane and 1,3-dithiolane, to afford the compound of formula U.

The preparation of a compound of formula V may be achieved by a variety of synthetic methods, including but not limited to, by way of example, at least the step of reacting a compound of formula Ar¹⁰X with a compound of formula YAr⁹, or a compound of formula Ar¹⁰Y with a compound of formula XAr⁹, in the presence of a suitable cross-coupling catalyst of palladium(0), palladium(II), palladium (IV), nickel(0), nickel(II), or nickel(IV), wherein X is a leaving group selected from the group consisting of Cl, Br, I, OTs, OTf, OMs, or OH, and Y is selected from the group consisting of SnR³, wherein R is selected from the group consisting of hydrogen, linear or branched C₁-C₂₄ alkyl, substituted or unsubstituted C₆-C₂₄ aryl, substituted or unsubstituted C₇-C₂₅ arylalkyl (the Migita-Kosugi-Stille reaction, as taught in M. Kosugi, K. Sasazawa, Y. Shikizu, T. Migita, Chemistry Letters, 1977, 6, 301-302; D. Milstein, J. K. Stille, Journal of the American Chemical Society 1978, 100, 3636-3638), ZnX′, wherein X′ is selected from the group consisting of Cl, Br, or I (the Negishi reaction, as taught in A. O. King, N. Okukado, E. Negishi, Journal of the Chemical Society, Chemical Communications 1977, 19, 683; E. Negishi, Accounts of Chemical Research 1982, 15, 340-348), or BXn, wherein BXn is selected from the group consisting of (i) boronic acid, or (ii) an organoborane, including but not limited to 9-borabicyclo[3.3.1]nonane, disiamylborane, diisoproylborane, or dicyclohexylborane, or (iii) a boronic ester, including but not limited to those derived from pinacol, neopentyl glycol, 2,3-butanediol, ethylene glycol, 1,3-propanediol, 2-methyl-1,3-propanediol, 2,4-pentanediol, 2,4-dimethyl-2,4-pentanediol, 2,2-diethyl-1,3-propanediol or catechol, or (iv) a boronate, including but not limited to sodium trihydroxyboronate, lithium triisopropylboronate, or cyclic triol boronates, or (v) N-coordinated boronate, including but not limited to those derived from diethanolamine, N-methyldiethanolamine, N-phenyldiethanolamine, or N-methyliminodiacetic acid, or (vi) triflic borates such as potassium trifluoroborate (the Suzuki-Miyaura reaction, as taught in N. Miyaura, A. Suzuki, Chemical Reviews 1995, 95, 2457-2483), or by nucleophilic acyl substitution with an organometallic reagent of the formula Ar⁹M′X or Ar¹⁰M′X, wherein M′ is an electropositive metal as Li, Na, or Mg, which is generated in situ in the presence of a metal M′ or an organometallic reagent RM′X′, wherein R is selected from the group consisting of hydrogen, linear or branched C₁-C₂₄ alkyl, substituted or unsubstituted C₆-C₂₄ aryl, or substituted or unsubstituted C₇-C₂₅ arylalkyl and X′ is selected from the group consisting of Cl, Br, or I, and then coupled to the corresponding compound of formula Ar¹⁰X or Ar⁹X, respectively, wherein Ar⁹ and Ar¹⁰ are as defined above.

### Example 6—Process for the Synthesis of 4-(4-(3,3,4,4,5,5-hexafluoro-2-(5-methyl-2-phenylthiazol-4-yl)cyclopent-1-en-1-yl)-5-methylthiophen-2-yl)phthalaldehyde

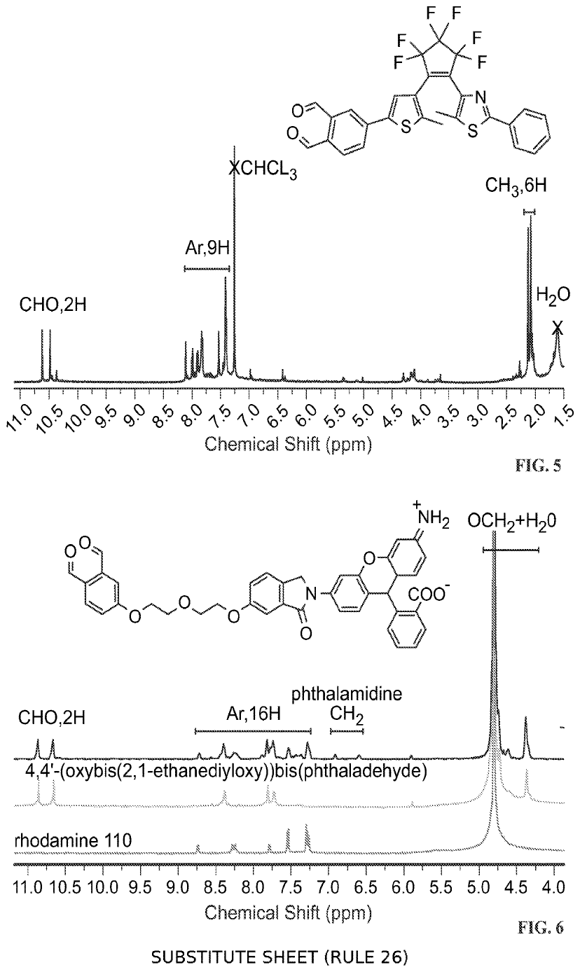

3-bromo-2-methylthien-5-ylboronic acid (2.35 g, 11 mmol) and 4-bromo-ortho-phthalaldehyde (2.42 g, 11 mmol) were combined with palladium (0) tetrakis(triphenylphosphine) (0.22 g) and aqueous 2M sodium carbonate (50 mL) in tetrahydrofuran (120 mL) in a 500 ml flask and refluxed overnight. The reaction was combined with water (100 mL) and diethyl ether (100 mL), then the organic layer was separated and dried over MgSO₄, filtered through filter paper, and the solvent was removed by rotary evaporation. Addition of ether to the residue produced an insoluble powder that was collected by filtration and washed with ether. Purification by column chromatography on SiO₂ in 6:1 hexanes:ethyl acetate eluent gave the intermediate product 4-(4-bromo-5-methyl-2-thienyl)phthalaldehyde (2.4 g, 70%), of which 1.9 grams was protected with ethylene glycol (4.5 g, 73 mmol) in refluxing benzene (100 mL) in the presence of catalytic p-toluenesulfonic acid (12 mg) under a Dean-Stark apparatus for 22 h to remove the water byproduct. The reaction mixture was washed with aqueous 2M sodium bicarbonate (100 mL), then dried over MgSO₄ (10 g), filtered through filter paper, and the solvent was removed by rotary evaporation in vacuum to yield the protected intermediate 2-(3,4-di(1,3-dioxolan-2-yl)phenyl)-4-bromo-5-methylthiophene as yellow crystals (2.0 g, 78%), which were dissolved anhydrous THF (100 mL) in a dry flask under Ar. A solution of 1.6M n-BuLi in hexane (3.8 mL) was added dropwise at −78° C. over 1 h. After the mixture stirred for 30 min, 1.85 grams of 1-(2-methyl-5-phenyl-3-thiazolyl) perfluorocyclopentene, prepared as taught in WO2008072419 A1, in anhydrous THF (10 mL) was added dropwise. The reaction was further stirred at −78° C. for 2 h, and the reaction mixture was allowed to slowly warm to the room temperature. The reaction was quenched with distilled water, extracted with diethyl ether, dried over MgSO₄, and concentrated under reduced pressure. The crude product was purified by column chromatography on silica gel using 5:1 hexanes:ethyl acetate eluent to afford 4-(2-(5-(3,4-di(1,3-dioxolan-2-yl)phenyl)-2-methylthiophen-3-yl)-3,3,4,4,5,5,-hexafluorocyclopent-1-en-1-yl)-5-methyl-2-phenylthiazole (2 g, 60%), which was deprotected in a 5:1 mixture of acetone and water with 2 equivalents of para-toluenesulfonic acid under reflux overnight at 60° C. Purification by column chromatography on SiO₂ in 6:1 hexanes:ethyl acetate eluent afforded 4-(4-(3,3,4,4,5,5-hexafluoro-2-(5-methyl-2-phenylthiazol-4-yl)cyclopent-1-en-1-yl)-5-methylthiophen-2-yl)phthalaldehyde (0.33 g, 19%). To validate the identity of the compound, a ¹H NMR spectrum of 4-(4-(3,3,4,4,5,5-hexafluoro-2-(5-methyl-2-phenylthiazol-4-yl)cyclopent-1-en-1-yl)-5-methylthiophen-2-yl)phthalaldehyde in CDCl₃ is given in FIG. 5.

In some embodiments, the presently claimed invention is directed at compounds of formula W, selected from the group consisting of formulae W1 or W2,

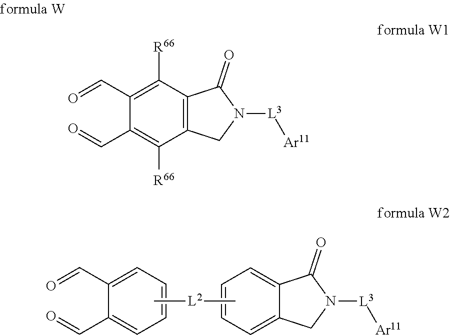

wherein R⁶⁶ and R⁶⁷ are independently of each other selected from the group consisting of hydrogen, linear or branched C₁-C₂₄ alkyl, substituted or unsubstituted C₇-C₂₅ arylalkyl, halogen, OH, or OR, wherein R is selected from the group consisting of hydrogen, linear or branched C₁-C₂₄ alkyl, substituted or unsubstituted C₆-C₂₄ aryl, substituted or unsubstituted C₇-C₂₅ arylalkyl, L² is an optional linking unit connecting a 3- or 4-substituted ortho-phthalaldehyde moiety to a 3-, 4-, 5-, or 6-substituted phthalimidine moiety in the compound of formula W2, comprising either a moiety of formula J, selected from the group consisting of formulae J1, J2, J3, J4, J5, J6, J7, J8, J9, J10, J11, J12, J13, J14, J15, or J16,

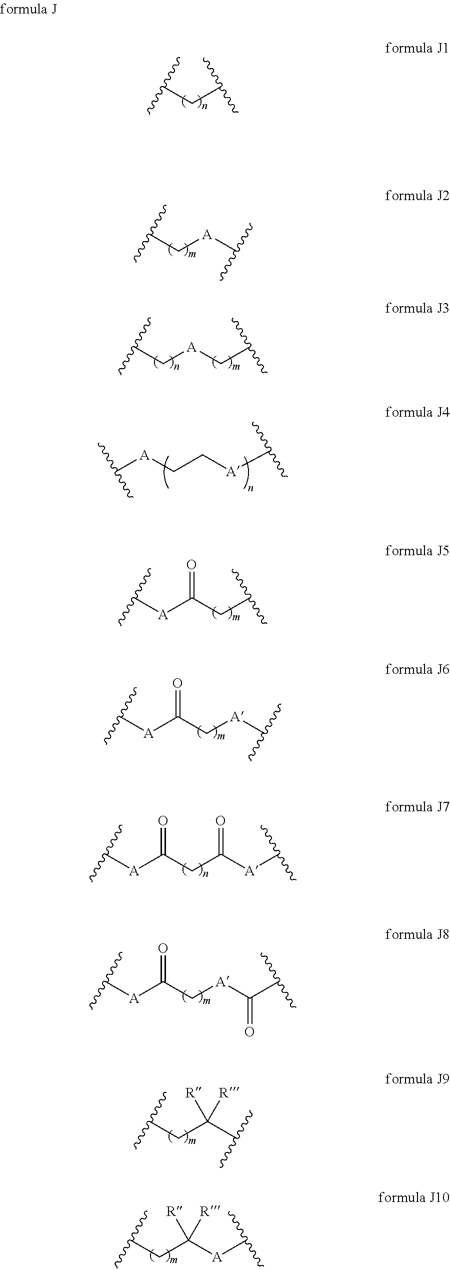

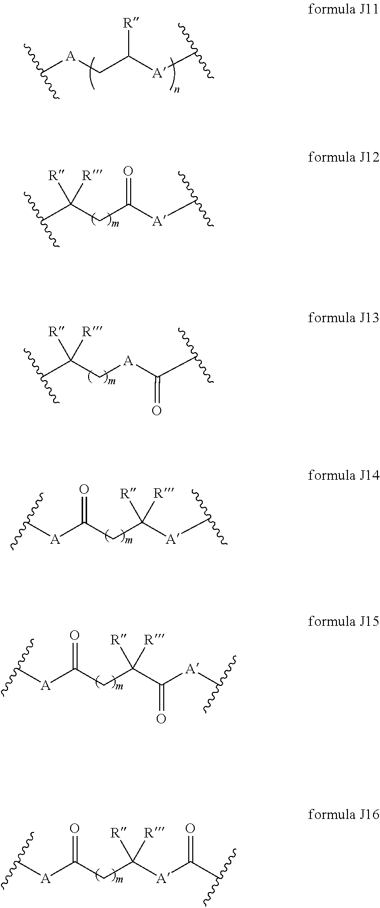

or a 1,2-, 1,3-, or 1,4-substituted phenylene moiety of formula K, selected from the group consisting of formulae K1, K2, K3, K4, K5, K6, K7, K8, K9, K10, K11, K12, K13, K14, or K15,

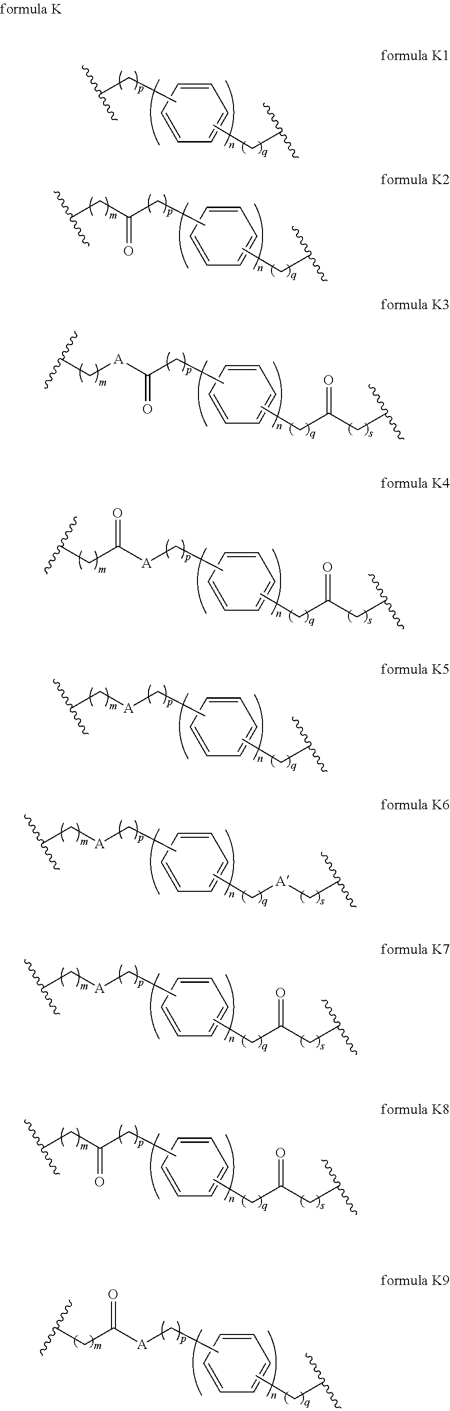

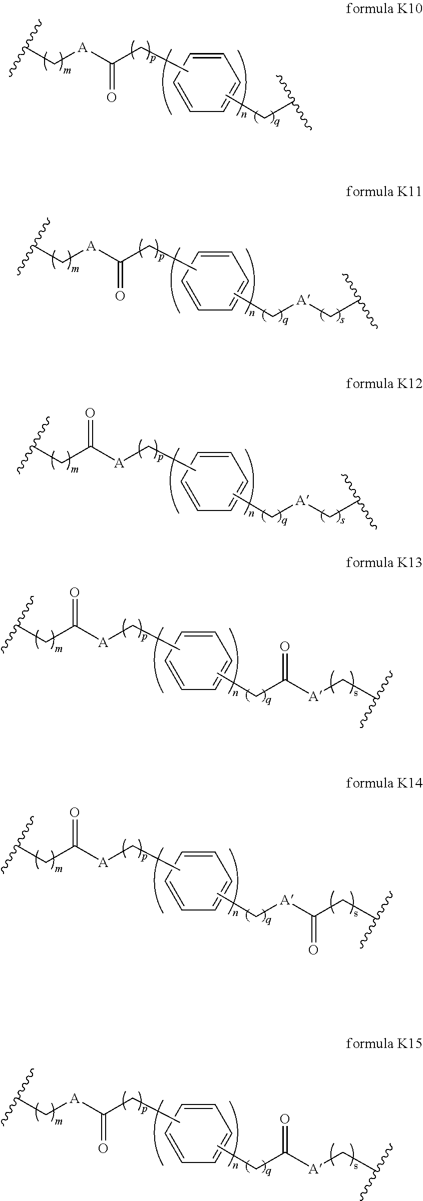

L³ is an optional linking unit, which when present in the compound of formula W, possesses a carbon atom bonded directly to the phthalimidine nitrogen atom, comprising a moiety selected from the group of formulae J1, J3, J9, J10, J12, or J13,

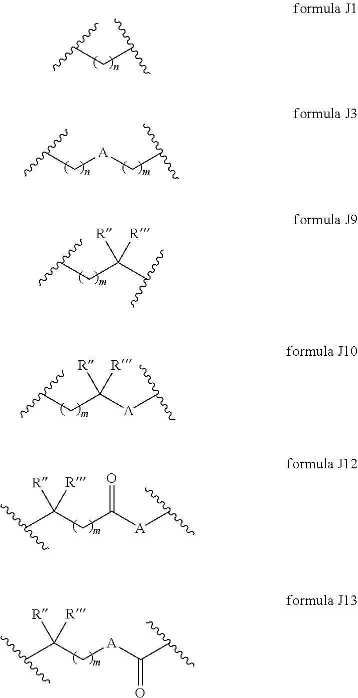

wherein A and A′ are, independently of each other, linking units selected from the group consisting of O, NH, S, SO₂, O(C═O), NH(C═O), NH(C═S), or S(C═O), R″ and R′″ are independently of each other selected from the group consisting of hydrogen, linear or branched C₁-C₂₄ alkyl, substituted or unsubstituted C₆-C₂₄ aryl, substituted or unsubstituted C₇-C₂₅ arylalkyl, C(═O)R, C(═O)OR, or OR, wherein R is selected from the group consisting of hydrogen, linear or branched C₁-C₂₄ alkyl, substituted or unsubstituted C₆-C₂₄ aryl, substituted or unsubstituted C₇-C₂₅ arylalkyl, n denotes a repeating unit that may number from 1 to 24, m, p, q, and s independently of each other denote repeating units that may number from 0 to 24, which may be either monodisperse or polydisperse, and wherein the O(C═O), NH(C═O), NH(C═S), or S(C═O) moieties of A and A′, or the unsymmetrical linking units of formula J or formula K may otherwise be oriented in either of the two possible directions, and Ar¹¹ is an ultraviolet absorber moiety of formula I as defined above,
or a visible chromophore moiety derived from a commercially available chromophore compound possessing a primary amine in its constitution, including but not limited to disperse black 1 (CI 11365), disperse black 2 (CI 11255), food black 2 (CI 27755), disperse black 3 (CI 11025), disperse black 5 (CI 11220), disperse black 6 (CI 37235), disperse black 7 (CI 11035), reactive black 8 (CI 18207), disperse black 9 (CI 111301), direct black 10 (CI 31545), direct black 11 (CI 30240), direct black 13 (CI 30025), direct black 14 (CI 30345), direct black 15 (CI 22620), direct black 17 (CI 27700), acid black 18 (CI 27790), direct black 19 (CI 35255), direct black 20 (CI 30395), acid black 20 (CI 27065), direct black 21 (CI 31580), direct black 22 (CI 35435), acid black 23 (CI 27230), disperse black 26, direct black 27 (CI 31810), direct black 28 (CI 35260), direct black 29 (CI 22580), disperse black 29, direct black 30 (CI 23675), disperse black 30, direct black 32 (CI 35440), direct black 34 (CI 35075), direct black 36 (CI 31665), direct black 38 (CI 30235), direct black 48 (CI 34000), direct black 51 (CI 27720), direct black 56 (CI 34170), acid black 66 (CI 30275), amido black 10B (CI 20470), basic brown 1 (CI 21000), bismarck brown R (CI 21010), C.I. food brown 1, mordant brown 33 (CI 13250), direct brown 53 (CI 13320), congo red (CI 22120), basic red 2 (CI 50240), disperse red 4 (CI 60755), basic red 5 (CI 50040), basic red 9 (CI 42500), vat red 10 (CI 67000), disperse red 11 (CI 62015), vat red 28 (CI 65710), D&C red 33 (CI 17200), disperse red 53 (CI 60759), disperse red 60 (CI 60756), disperse red 86 (CI 62175), disperse red 91 (CI 60753), disperse red 92 (CI 60752), basic orange 1 (CI 11320), basic orange 2 (CI 11270), disperse orange 3 (CI 11005), solvent orange 4 (CI 11320:1), basic orange 5 (CI 46035), direct orange 6 (CI 23365 and 23375), disperse orange 7 (CI 11240), direct orange 8 (CI 22130 and 22140), disperse orange 11 (CI 60700), basic orange 15 (CI 46045), direct orange 24 (CI 20130), acriflavine (CI 4600), acridine yellow G (CI 46025), aniline yellow (CI 11000), fast yellow AB (CI 13015), reactive yellow 3 (CI 13245), direct yellow 7 (CI 49010), disperse yellow 9 (CI 10375), azure C (CI 49410), brilliant cresyl blue (CI 51010), toluidine blue (CI 52040), tryptan blue (CI 23850), toluylene blue (CI 49410), water blue (CI 42755), disperse blue 1 (CI 64500), reactive blue 4 (CI 61205), disperse blue 9 (CI 61115), basic blue 16 (CI 12210), reactive blue 19 (CI 61200), acid blue 45 (CI 63010), vat blue 64 (CI 66730), disperse blue 73 (CI 63265), disperse blue 81 (CI 63603), acid blue 129 (CI 62058), basic violet 2 (CI 42520), disperse violet 17 (CI 60712), acid violet 19 (CI 42685), disperse violet 26 (CI 62025) disperse violet 28 (CI 61102), acriflavine, proflavine, thionine, ortho-dianisidine, fuchsine, induline, mauveine, seminaphtharhodafluor, 5,10,15,20-tetrakis(4-aminophenyl) porphyrin, N-(4-aminophenyl) carbazole, rhodamine sulfate, rhodamine 110 chloride, rhodamine 123, 2-(4-aminophenyl)-6-methylbenzothiazole, 2-(4-aminophenyl)-6-methylbenzothiazole-7-sulphonic acid, 2-(4-aminophenyl)benzimidazole, 3-(8-methyl-imidazo[1,2-a]pyridin-2-yl)-phenylamine, 4-(5-methyl-2-benzoxazolyl)benzenamine, 4-(5-fluoro-2-benzothiazolyl)-2-methylbenzenamine, or 4-((4-nitrophenyl)azo)benzene-1,3-diamine.

In some embodiments, the presently claimed invention provides a process for the preparation of a compound of formula W, comprising at least the step of reacting at least one compound of formula X, selected from the group consisting of formulae X1, X2, or X3,

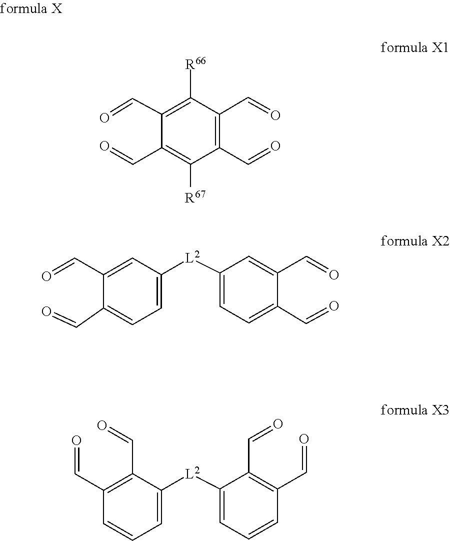

wherein R⁶⁶, R⁶⁷, and L² are as defined above, with less than one molar equivalent of a compound of formula H₂NL³Ar¹¹, wherein H₂NL³Ar¹¹ is either: an ultraviolet absorber compound of formula Y, selected from the group consisting of formulae Y1, Y2, or Y3,

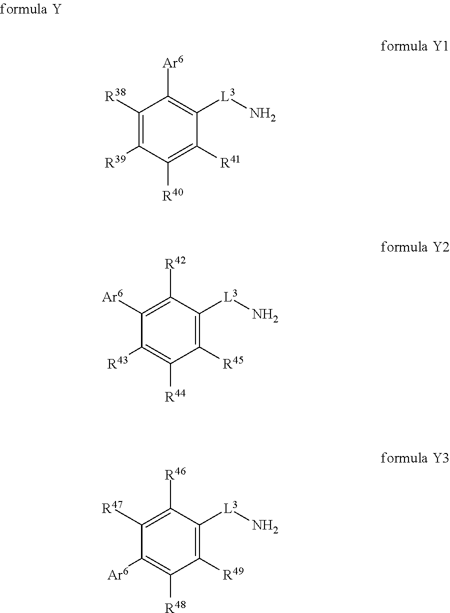

wherein Ar⁶, R³⁸, R³⁹, R⁴⁰, R⁴¹, R⁴², R⁴³, R⁴⁴, R⁴⁵, R⁴⁶, R⁴⁷, R⁴⁸, and R⁴⁹ are as defined above, L³ is an optional linking unit, which when present in the compound of formula Y, possesses a carbon atom bonded directly to the amine nitrogen atom, comprising a moiety selected from the group of formulae J1, J3, J9, J10, J12, or J13,

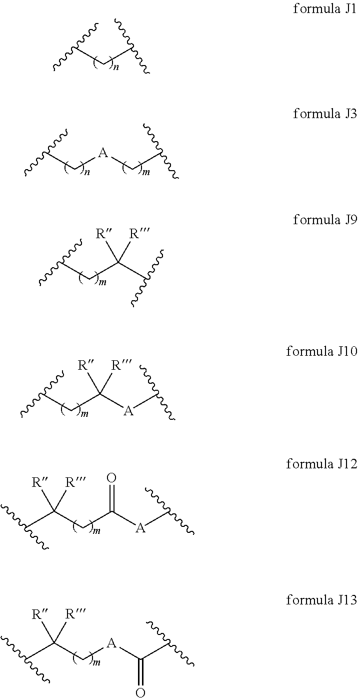

or H₂NL³Ar¹¹ is a commercially available, primary amine-functionalized visible chromophore, including but not limited to disperse black 1 (CI 11365), disperse black 2 (CI 11255), food black 2 (CI 27755), disperse black 3 (CI 11025), disperse black 5 (CI 11220), disperse black 6 (CI 37235), disperse black 7 (CI 11035), reactive black 8 (CI 18207), disperse black 9 (CI 111301), direct black 10 (CI 31545), direct black 11 (CI 30240), direct black 13 (CI 30025), direct black 14 (CI 30345), direct black 15 (CI 22620), direct black 17 (CI 27700), acid black 18 (CI 27790), direct black 19 (CI 35255), direct black 20 (CI 30395), acid black 20 (CI 27065), direct black 21 (CI 31580), direct black 22 (CI 35435), acid black 23 (CI 27230), disperse black 26, direct black 27 (CI 31810), direct black 28 (CI 35260), direct black 29 (CI 22580), disperse black 29, direct black 30 (CI 23675), disperse black 30, direct black 32 (CI 35440), direct black 34 (CI 35075), direct black 36 (CI 31665), direct black 38 (CI 30235), direct black 48 (CI 34000), direct black 51 (CI 27720), direct black 56 (CI 34170), acid black 66 (CI 30275), amido black 10B (CI 20470), basic brown 1 (CI 21000), bismarck brown R (CI 21010), C.I. food brown 1, mordant brown 33 (CI 13250), direct brown 53 (CI 13320), congo red (CI 22120), basic red 2 (CI 50240), disperse red 4 (CI 60755), basic red 5 (CI 50040), basic red 9 (CI 42500), vat red 10 (CI 67000), disperse red 11 (CI 62015), vat red 28 (CI 65710), D&C red 33 (CI 17200), disperse red 53 (CI 60759), disperse red 60 (CI 60756), disperse red 86 (CI 62175), disperse red 91 (CI 60753), disperse red 92 (CI 60752), basic orange 1 (CI 11320), basic orange 2 (CI 11270), disperse orange 3 (CI 11005), solvent orange 4 (CI 11320:1), basic orange 5 (CI 46035), direct orange 6 (CI 23365 and CI 23375), disperse orange 7 (CI 11240), direct orange 8 (CI 22130 and CI 22140), disperse orange 11 (CI 60700), basic orange 15 (CI 46045), direct orange 24 (CI 20130), acriflavine (CI 4600), acridine yellow G (CI 46025), aniline yellow (CI 11000), fast yellow AB (CI 13015), reactive yellow 3 (CI 13245), direct yellow 7 (CI 49010), disperse yellow 9 (CI 10375), azure C (CI 49410), brilliant cresyl blue (CI 51010), toluidine blue (CI 52040), tryptan blue (CI 23850), toluylene blue (CI 49410), water blue (CI 42755), disperse blue 1 (CI 64500), reactive blue 4 (CI 61205), disperse blue 9 (CI 61115), basic blue 16 (CI 12210), reactive blue 19 (CI 61200), acid blue 45 (CI 63010), vat blue 64 (CI 66730), disperse blue 73 (CI 63265), disperse blue 81 (CI 63603), acid blue 129 (CI 62058), basic violet 2 (CI 42520), disperse violet 17 (CI 60712), acid violet 19 (CI 42685), disperse violet 26 (CI 62025) disperse violet 28 (CI 61102), acriflavine, proflavine, thionine, ortho-dianisidine, fuchsine, induline, mauveine, seminaphtharhodafluor, 5,10,15,20-tetrakis(4-aminophenyl)porphyrin, N-(4-aminophenyl)carbazole, rhodamine sulfate, rhodamine 110 chloride, rhodamine 123, 2-(4-aminophenyl)-6-methylbenzothiazole, 2-(4-aminophenyl)-6-methylbenzothiazole-7-sulphonic acid, 2-(4-aminophenyl)benzimidazole, 3-(8-methyl-imidazo[1,2-a]pyridin-2-yl)-phenylamine, 4-(5-methyl-2-benzoxazolyl)benzenamine, 4-(5-fluoro-2-benzothiazolyl)-2-methylbenzenamine, or 4-((4-nitrophenyl)azo)benzene-1,3-diamine,
in a solution containing water and/or alcohols such as methanol, ethanol, or isopropanol, or combinations thereof, optionally mixed with other alcohols or organic solvents, which may be selected from the group including but not limited to dimethylsulfoxide, N,N-dimethylformamide, tetrahydrofuran, 3-methyltetrahydrofuran, 1,4-dioxane, acetonitrile, acetone, ethyl acetate, ethyl lactate, and the like, such that the aqueous or alcoholic solution can dissolve both reactants of formula X and Y, such that the two ortho-phthalaldehyde moieties of the compound of formula X are consumed only partially in the reagent-free phthalimidine-forming reaction with H₂NL³Ar¹¹, rendering the unreacted ortho-phthalaldehyde moieties in the product compound available for reaction with proteins in skin tissue.

An advantage of this process for the preparation of the ortho-phthalaldehyde ultraviolet absorber or visible chromophore is that it enables the target compound to be generated in situ in a cosmetically acceptable aqueous or alcoholic solution, in some embodiments, which can subsequently be employed as a topical composition with minimal or no further processing, and avoiding the need for further purification techniques.

### Example 7—Process for the Synthesis of 2-(6-(6-(2-(2-(3,4-diformylphenoxy)ethoxy)ethoxy)-1-oxoisoindolin-2-yl)-3-iminio-3H-xanthen-9-yl)benzoate

1 mg of 4,4′-(oxybis(2,1-ethanediyloxy))bis(phthalaldehyde), prepared as taught in CN110981715 and J.-H. Wang, Y.-L. Tang, Z. Gong, R. Jain, F. Xiao, Y. Zhou, D. Tan, Q. Li, N. Huang, S.-Q. Liu, K. Ye, C. Tang, M.-Q. Dong, X. Lei, Nature Communications 2022, 13, 1468, was combined with 1 mg of rhodamine 110 in 1 ml of a 1:2 v/v mixture of D₂O and CD₃CN and held at ambient temperature for 60 minutes, affording 2-(6-(6-(2-(2-(3,4-diformylphenoxy)ethoxy)ethoxy)-1-oxoisoindolin-2-yl)-3-iminio-3H-xanthen-9-yl)benzoate as the predominant product. To validate the identity of the compound, a graph of the ¹H NMR spectrum of 2-(6-(6-(2-(2-(3,4-diformylphenoxy)ethoxy)ethoxy)-1-oxoisoindolin-2-yl)-3-iminio-3H-xanthen-9-yl)benzoate in D₂O:CD₃CN (1:1 v/v) is plotted in FIG. 6.

### Compositions of Ortho-Phthalaldehyde Ultraviolet Absorbers and Visible Chromophores for Topical Use on Human or Other Mammalian Skin

In some embodiments, the presently claimed invention provides compositions of solutions, gels, or emulsions containing an ortho-phthalaldehyde-functionalized ultraviolet-absorbing agent or visible chromophore agent suitable for application to skin. Suitable product forms include lotions, creams, gels, sticks, sprays, ointments, and mousses. When the ortho-phthalaldehyde compound in the composition is an ultraviolet-absorbing agent, the composition is considered to be a semi-permanent sunscreen. When the ortho-phthalaldehyde compound in the composition is a visible chromophore agent, the composition is considered to be a semi-permanent topical tattoo ink.

In order to bond any of ortho-phthalaldehyde compounds of formula A, formula G, or formula U to proteins in the stratum corneum of skin tissue, the compounds are first dissolved or dispersed in a solution, gel, or emulsion comprising, in some embodiments, water or alcohol, mixtures thereof, and optionally, cosmetic oils, silicone fluids, or mixtures thereof, with optional modifiers in the form of additives selected from the group consisting of rheological modifiers such as thickeners or thixotropic agents, surfactants or emulsifiers, emollients, humectants, film formers, antimicrobial agents, or washable dyes.

In some embodiments, the ortho-phthalaldehyde-functionalized ultraviolet absorber or visible chromophore of formula A, formula G, or formula U is dissolved at a concentration of greater than 1% by weight in a solvent mixture containing alcohol or water, or a mixture thereof, and optionally, saline, phosphate buffer, organic solvents, oils, or silicone fluids,
wherein suitable alcohols include but are not limited to ethanol, isopropanol, 1,3-propanediol, propylene glycol, 1-propanol, 1,3-butanediol, 1,4-butanediol, glycerol, stearyl alcohol, cetyl alcohol, oleyl alcohol, 1-hexanol, 1-heptanol, 1-decanol, 1,3-dioxolane, 1,6-hexanediol, 2-butoxyethanol, 2-(2-butoxyethanol), 2-(2-ethoxy)ethyl acetate, 2-(2-ethoxyethoxy)ethanol, 2-(2-methoxyethoxy)ethanol, 2-butanone, 2-butoxyethyl acetate, 2-ethoxyethanol, 2-ethoxyethyl acetate, 2-methoxyethanol, and geraniol, amyl acetate, butyl methyl ketone, or 1,10-decanediol,
suitable organic solvents include but are not limited to ethyl acetate, isopropyl myristate, isopropyl stearate, coco-caprylate, squalene, n-decane, n-hexadecane, dodecane, lauraldehyde, n-octane, (+/−)-limonene, p-cymene, methyl laurate, ethyl oleate, 2-furancarboxyaldehyde, amyl alcohol, decafluoropentane, acetone, or isododecane, suitable oils include but are not limited to açaí oil, agar oil, ajwain oil, algal oil, almond oil, amaranth oil, angelica root oil, apple seed oil, apricot oil, anise oil, argan oil, artichoke oil, astrocaryum murumuru butter, attar, avocado oil, babassu oil, basil oil, bay leaf oil, beech nut oil, ben oil, bergamot oil, birch oil, bitter gourd oil, blackcurrant seed oil, black seed oil, bladderpod oil, borage seed oil, Borneo tallow nut oil, bottle gourd oil, Brazil nut oil, buffalo gourd oil, burdock oil, buriti oil, butternut squash seed oil, calamus oil, camphor oil, candlenut oil, cannabis flower oil, canola oil, cape chestnut oil, caraway seed oil, cardamom seed oil, carob pod oil, carrot seed oil, cashew oil, castor oil, cedar oil, chamomile oil, cinnamon oil, citronella oil, clary sage oil, clove oil, cocklebur oil, cocoa butter, coconut oil, cocoa butter, cohune oil, colza oil, coriander seed oil, corn oil, cottonseed oil, cypress oil, date seed oil, dika oil, egusi seed oil, evening primrose oil, eucalyptus oil, false flax oil, fennel seed oil, flaxseed oil, frankincense oil, grape seed oil, grapefruit seed oil, hazelnut oil, hemp oil, hickory nut oil, Jamaican cobnut oil, jasmine oil, jojoba oil, kapok seed oil, kenaf seed oil, lallemantia oil, lavender oil, lemon oil, lemongrass oil, linseed oil, orange oil, macadamia oil, mafura oil, mango oil, marula oil, meadowfoam seed oil, mongogo nut oil, mowrah butter, mustard seed oil, olive oil, Niger seed oil, nutmeg butter, ojon oil, okra seed oil, palm oil, palo santo oil, papaya seed oil, parsley oil, peanut oil, pecan oil, peppermint oi perilla seed oil, persimmon seed oil, pequi oil, pili nut oil, pine nut oil, pistachio oil, pomegranate seed oil, poppyseed oil, pracaxi oil, prune kernel oil, pumpkin seed oil, quinoa oil, radish oil, ramtil oil, rapeseed oil, rice bran oil, royle oil, rose oil, rosehip oil, rosewood oil, rubber seed oil, sacha inchi oil, safflower oil, sage oil, salicornia oil, sandalwood oil, sapote oil, sassafras oil, savory oil, seje oil, shea butter, soybean oil, spruce oil, sunflower oil, tall oil, tangerine oil, taramira oil, tea seed oil, tea tree oil, thistle oil, thyme oil, tigernut oil, tobacco seed oil, tomato seed oil, tonka bean oil, walnut oil, watermelon seed oil, wintergreen oil, wheat germ oil, or yarrow oil,
suitable silicone fluids include but are not limited to dimethicone, cyclomethicone, caprylyl methicone, decamethyltetrasiloxane, decamethylcyclopentasiloxane, cycloethoxymethicone, cycloheptasiloxane, cyclohexasiloxane, dimethiconol, phenyl trimethicone, or cyclotrisiloxane, or any combinations thereof, with other optional modifiers, including but not limited to:
rheological modifiers such as (i) thickening agents, including natural gums such as xanthan gum, gum arabic, carrageenan, tragacanth gum, guar gum, tara gum, locust bean gum, agar agar, konjac gum, gellan gum, polyacrylates, poly(ethylene glycol) (PEG) and derivatives thereof such as PEG stearates, or (ii) thixotropic agents such as colloidal silica, bentonite, magnesium aluminum silicate, or polymerized vegetable oil, to modify how the composition flows under gravitational or shear forces,
biocompatible surfactants such as TWEEN 20, TWEEN 40, TWEEN 60, TWEEN 80, polysorbates such as polysorbate 20, polysorbate 40, polysorbate 60, polysorbate 80, decyl glucoside, sorbitan monolaurate, sodium lauryl sulfate, cocamidopropyl betaine, glyceryl stearate, PEG-100 stearate, sodium cocoamphoacetate, sodium cocoyl isethionate, sodium methyl cocoyl taurate, disodium laureth sulfosuccinate, sodium or lauroyl sarcosinate, and the like, to stabilize an emulsion, in some embodiments, and/or modify surface tension, and/or modify sedimentation properties when any components of the composition are present in suspension,
film-forming agents such as polyacrylates, acrylate copolymers, carbomers, polyvinylpyrrolidone, polyvinyl alcohol, polyethers, silicone resins, cellulose or cellulose derivatives such as ethylcellulose, hydroxyethylcellulose, polyurethanes, polyesters, polyquaterniums, polymethyl methacrylate, and natural resins, to protect the topical application and/or improve the efficiency of the phthalimidine-forming reaction of the ortho-phthalaldehyde ultraviolet absorber or visible chromophore agent,
emollients such as petrolatum, mineral oil, lanolin, and other naturally-derived cosmetic oils listed above, C₁₄-C₂₂ alkanes, C₉-C₁₆ isoparaffins, caprylic triglycerides, lauric triglycerides, cetyl salicylate, or soluble collagen, to soften or smooth the skin upon topical application of the composition,
humectants such as glycerol, hyaluronic acid, propylene glycol, sorbitol, butylene glycol, urea, alpha hydroxy acids such as lactic acid, glycolic acid, tartaric acid, citric acid, or mandelic acid, sodium pyrrolidone carboxylic acid, sodium hyaluronate, polyethylene glycol (PEG), honey, aloe vera, panthenol, sodium lactate, or betaine, to moisturize the skin upon topical application of the composition, antiseptics/antimicrobials such as benzalkonium chloride, benzethonium chloride, chlorhexidine, triclosan, phenoxyethanol, ethylhexylglycerin, benzoyl peroxide, silver compounds such as silver sulfadiazine, iodopropynyl butylcarbamate, or zinc pyrithione, to protect against bacterial contamination,
or washable dyes that are safe for topical use on skin, such as FD&C Red No. 40 (Allura Red), FD&C Yellow No. 5 (Tartrazine), FD&C Blue No. 1 (Brilliant Blue), D&C Red No. 27 (Erythrosine), D&C Red No. 7 (Acid Red), D&C Yellow No. 10 (Quinoline Yellow), beetroot extract, turmeric extract, or spirulina extract, to provide a temporary visible indication of topical coverage on the skin that can be washed away after development of the phthalimidine-forming reaction of the ortho-phthalaldehyde ultraviolet absorbing agent.

### Example 8—Composition of a Semi-Permanent Topical Tattoo Ink Containing 3,6-dihydroxy-4,5-dimethyl-1,2-benzenedicarboxaldehyde as a Visible Chromophore Agent

20 mg of 3,6-dihydroxy-4,5-dimethyl-1,2-benzenedicarboxaldehyde was mixed directly into 230 mg of a gel comprising 79.715% v/v water, 20% v/v ethanol, and 0.285% v/v carbomer 940 and stirred mechanically until a homogenous gel was obtained.

### Example 9—Composition of a Semi-Permanent Photochromic Topical Tattoo Ink Solution Containing 4-[3,3,4,4,5,5-hexafluoro-2-[2-methyl-5-(3,4-diformylphenyl)-3-thienyl]-1-cyclopenten-1-yl]-5-methyl-2-phenylthiazole as a Photochromic Visible Chromophore Agent

4-[3,3,4,4,5,5-hexafluoro-2-[2-methyl-5-(3,4-diformylphenyl)-3-thienyl]-1-cyclopenten-1-yl]-5-methyl-2-phenylthiazole (20 mg) was dissolved in ethyl acetate (60 mg) to obtain a 25% w/w solution. 10 μl of this concentrated solution of the photochromic ortho-phthalaldehyde visible chromophore agent was mixed with dimethicone (10 μl), isopropyl myristate (10 μl), and LexFilm Spray polyester-10+propylene glycol dibenzoate.

### Methods of Topical Application of Ortho-Phthalaldehyde Ultraviolet Absorber or Visible Chromophore Compositions to Form Semi-Permanent Bonds to Human or Other Mammalian Skin Tissue

The compositions of the presently claimed invention may be employed for various end-uses, such as recreational or daily-use sunscreens, cosmetics/make-up, anti-aging products, body art, or combinations thereof. In some embodiments, the presently claimed invention provides methods of topical application of solutions, gels, or emulsions of ortho-phthalaldehyde-functionalized compounds in the form of lotions, creams, gels, sticks, sprays, ointments, or mousses to the skin in order to facilitate the reaction of compounds of formula A, formula G, or formula U with endogenous proteins in skin tissue to form phthalimidine linkages that cause the ultraviolet-absorbing agent or visible chromophore agent in the composition to remain bonded to the skin tissue as a semi-permanent mark, for a period of days to weeks until the cells of the strateum corneum are shed. The term “semi-permanent” is meant to convey that the ultraviolet-absorbing agent or visible chromophore agent is bonded covalently and, in a practical sense, irreversibly, to proteins associated with skin cells of the stratum corneum, such that the ultraviolet-absorbing agent or visible chromophore agent cannot be removed from the skin without the removal of the skin cells in the stratum corneum to which they are bonded.

In some embodiments, the topical solution may be applied manually using droplets or jets generated from a suitable bottle, pipette, or syringe.

In some embodiments, the topical solution may be applied manually using conventional handheld tools for transferring colorants, such as brushes, pens, and markers.

In some embodiments, the topical solution may be applied with the assistance of a thin-film applicator in which designs or patches of the topical product are applied or printed to a flexible backing layer of paper, plastic, or fabric with appropriate adhesives and alcoholic solvents to render the compounds soluble at the skin-applicator interface, in a manner akin to those taught in U.S. Pat. No. 10,143,641B2.

### Example 10—Method of Topical Application of a Semi-Permanent Topical Tattoo Ink Containing 3,6-dihydroxy-4,5-dimethyl-1,2-benzenedicarboxaldehyde as the Visible Chromophore Agent

Freshly excised porcine skin was allowed to hydrate in reverse-osmosis purified water for 4 h, then it was cut into rectangular samples and stored for 24 hours in a closed container on top of a paper towel saturated with a solution of 90% water and 10% glycerol. The stratum corneum was wiped with a cotton gauze pad saturated with an aqueous solution of lysine at a concentration of 2 wt %. The gel composition prepared as described in Example 9 was scooped onto a spatula, which was used to apply a thin layer of the gel in a circle of approximately 1-cm diameter on the stratum corneum. After allowing the gel to dry on the skin for 10 minutes, the skin sample was returned to the closed container with the saturated paper towel to allow the skin to remain hydrate while the reaction continued to develop. After 3 hours, the skin was removed and washed liberally by scrubbing the skin with a loofah saturated with a soap solution of 10% alcanox in water until a foam lather was formed, rinsed with reverse-osmosis water, and wiped with a paper towel saturated with a 70% isopropanol solution, and finally patted dry with a paper towel. This washing procedure was repeated five times.

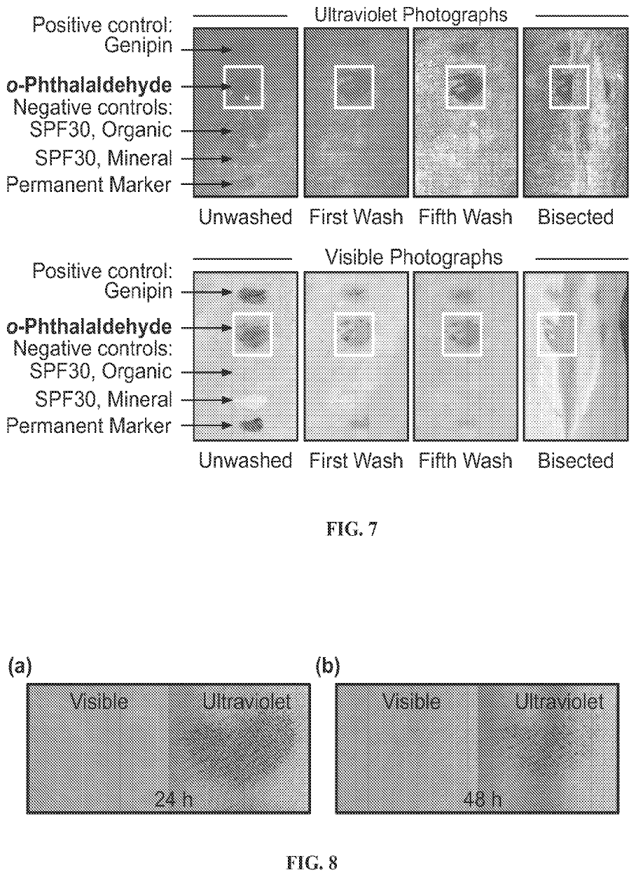

For the purposes of demonstrating the semi-permanent retention of the visible mark rendered on the porcine skin by the ortho-phthalaldehyde visible chromophore agent of the composition prepared as described in Example 8 and administered by the method described in Example 10, several comparative examples in the form of negative and positive control materials were applied topically adjacent to the semi-permanent topical tattoo and subjected to the same washing conditions. Photographs recorded before and after the washing procedure are annotated in FIG. 7, demonstrating that the topical tattoo rendered by the 3,6-dihydroxy-4,5-dimethyl-1,2-benzenedicarboxaldehyde visible chromophore agent of the composition is retained along with the genipin-based positive control, whereas the negative controls lacking a moiety that can bond to endogenous skin proteins were removed after the first wash. A photograph of the excessively washed skin sample, after having been bisected through to the subcutis with a knife and folded open to expose the skin's layers, demonstrates that the ortho-phthalaldehyde visible chromophore of this composition and method was localized in the stratum corneum and did not penetrate to lower epidermal or dermal layers of the skin.

### Example 11—Methods of Topical Application of a Semi-Permanent Photochromic Topical Tattoo Ink Solution Containing 4-[3,3,4,4,5,5-hexafluoro-2-[2-methyl-5-(3,4-diformylphenyl)-3-thienyl]-1-cyclopenten-1-yl]-5-methyl-2-phenylthiazole as the Photochromic Visible Chromophore Agent

100 μL of the composition described in Example 10 was loaded into a squeeze-dropper and two drops of the ink were placed directly on untreated human skin in vivo from a height of approximately 1 mm. The remainder of the solution was applied to a paintbrush and transferred to an adjacent area of the skin by manually painting a mark in the shape of a heart. The marks rendered on the skin by the composition containing the photochromic ortho-phthalaldehyde visible chromophore agent were allowed to dry for 1 h before the skin was washed by scrubbing with handsoap and water in a sink in the manner of a typically daily routine.

Annotated photographs in FIG. 8 demonstrate that the composition prepared as described in Example 9 and applied topically as described in Example 11 is retained on the skin after a period of 48 hours, over which time the skin was subjected to ordinary activity, including showers, twice-daily soap-and-water scrubs, and light abrasion from clothing worn on top of the topical tattoo marks. Control markings of an analogous photochromic compound, which lacks the ortho-phthalaldehyde functionality, did not remain locally retained on the skin after the first soap scrub. This demonstration provides evidence that the ortho-phthaldehyde functions as intended to leave semi-permanent marks on the skin in vitro.

### Definitions

The term “administration” and variants thereof (e.g., “administering” a compound, “administering” a recombinant myxoma virus) in reference to a compound of the invention means introducing the compound into the system of the subject in need of treatment, such as via injection into the dermal layer of the skin of the subject. When a compound of the invention is provided in combination with one or more other active agents, “administration” and its variants are each understood to include concurrent and sequential introduction of the compound and other agents.

As used herein, the term “composition” is intended to encompass a product comprising the specified ingredients in the specified amounts, as well as any product which results, directly or indirectly, from combination of the specified ingredients in the specified amounts.

A “cosmetically acceptable” component is one that is suitable for use with humans and/or animals without undue adverse side effects (such as toxicity, irritation, and allergic response) commensurate with a reasonable benefit/risk ratio.

A “safe and effective amount” refers to the quantity of a component that is sufficient to yield a desired therapeutic response without undue adverse side effects (such as toxicity, irritation, or allergic response) commensurate with a reasonable benefit/risk ratio when used in the manner of this invention.

As used throughout the entire application, the terms “a” and “an” are used in the sense that they mean “at least one”, “at least a first”, “one or more” or “a plurality” of the referenced components or steps, unless the context clearly dictates otherwise. For example, the term “a cell” includes a plurality of cells, including mixtures thereof.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range.

As used herein, the term “comprising” is intended to mean that the products, compositions and methods include the referenced components or steps, but not excluding others. “Consisting essentially of” when used to define products, compositions and methods, shall mean excluding other components or steps of any essential significance. Thus, a composition consisting essentially of the recited components would not exclude trace contaminants and pharmaceutically acceptable carriers. “Consisting of” shall mean excluding more than trace elements of other components or steps.

Kits for practicing the methods of the invention are further provided. By “kit” is intended any manufacture (e.g., a package or a container) comprising at least one reagent, e.g., a pH buffer of the invention. The kit may be promoted, distributed, or sold as a unit for performing the methods of the present invention. Additionally, the kits may contain a package insert describing the kit and methods for its use. Any or all of the kit reagents may be provided within containers that protect them from the external environment, such as in sealed containers or pouches. The advantages set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

As used herein, “ultraviolet-absorbing agent” or “ultraviolet absorber” refers to a material or compound or a chemical moiety that absorbs radiation with a molar extinction coefficient of at least 1000 mol⁻¹ cm⁻¹ for at least one wavelength in the region of the ultraviolet spectrum between wavelengths of 280 nm and 400 nm.

As used herein, “visible chromophore” is intended to mean a material or compound or a chemical moiety that absorbs radiation with a molar extinction coefficient of at least about 1000 mol⁻¹ cm⁻¹ for at least one wavelength in the visible portion of the visible spectrum between wavelengths of 400 nm and 760 nm.

As used herein, “topical” is intended to mean a manner of applying a material or compound directly to the surface of skin on a human or other mammalian body.

As used herein, the term “semi-permanent” is intended to mean an irreversible bond that persists for as long as skin cells remain present in the stratum corneum.

As used herein, the term “topical tattoo” in intended to mean a semi-permanent visible mark on the skin.

All references cited in the present application are incorporated in their entirety herein by reference to the extent not inconsistent herewith.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall there between.

1. (canceled)

2. A compound of formula A, selected from the group consisting of formulae A1 or A2, 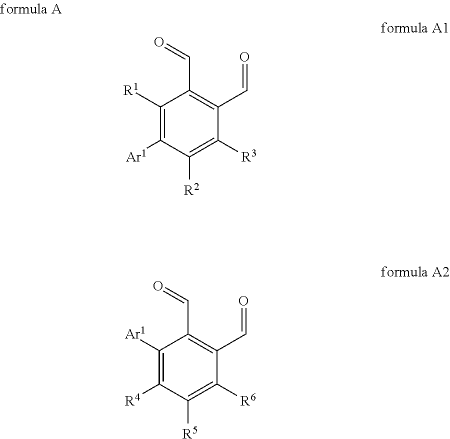
wherein Ar¹ is an ultraviolet absorbing moiety of formula B, formula C, formula D, or formula E, 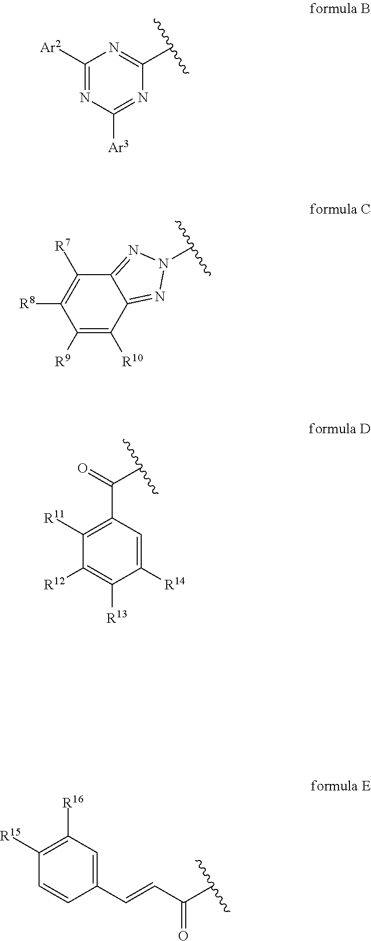
wherein Ar² and Ar³ are independently of each other a moiety of formula F, selected from the group consisting of formulae F1 or F2, 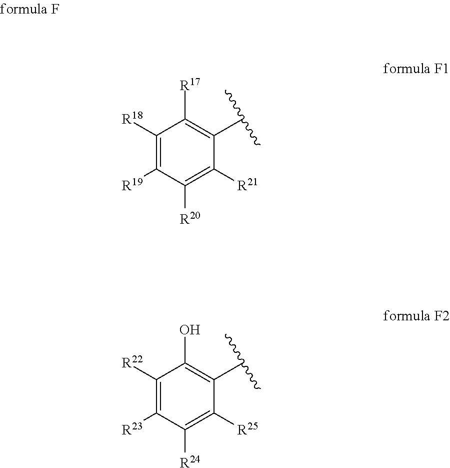
wherein R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, and R²⁵ are independently selected from the group consisting of hydrogen, linear or branched C₁-C₂₄ alkyl, linear or branched C₂-C₂₄ alkenyl, linear or branched C₂-C₂₄ alkynyl, substituted or unsubstituted C₅-C₂₄ cycloalkyl, substituted or unsubstituted C₆-C₂₄ aryl, substituted or unsubstituted C7-C25 arylalkyl, halogen, CHO, C(═O)R, C(═O)OR, C(═O)OM, CN, OR, NRR′, NO2, SR, S(═O)2R, S(═O)2OR, or S(═O)2OM wherein M is an alkali metal, and R and R′ are independently selected from the group consisting of hydrogen, linear or branched C1-C24 alkyl, substituted or unsubstituted C6-C24 aryl, substituted or unsubstituted C7-C25 arylalkyl, or R² and R³, together with the carbon atoms to which they are bonded, or R⁴ and R⁵, together with the carbon atoms to which they are bonded, or R⁵ and R⁶, together with the carbon atoms to which they are bonded, or R⁷ and R⁸, together with the carbon atoms to which they are bonded, or R⁸ and R⁹, together with the carbon atoms to which they are bonded, or R⁹ and R¹⁰, together with the carbon atoms to which they are bonded, or R¹¹ and R¹², together with the carbon atoms to which they are bonded, or R¹² and R¹³, together with the carbon atoms to which they are bonded, or R¹³ and R¹⁴, together with the carbon atoms to which they are bonded, or R¹⁵ and R¹⁶, together with the carbon atoms to which they are bonded, or R¹⁷ and R¹⁸, together with the carbon atoms to which they are bonded, or R¹⁸ and R¹⁹, together with the carbon atoms to which they are bonded, or R¹⁹ and R²⁰, together with the carbon atoms to which they are bonded, or R²⁰ and R²¹, together with the carbon atoms to which they are bonded, or R²² and R²³, together with the carbon atoms to which they are bonded, or R²³ and R²⁴, together with the carbon atoms to which they are bonded, or R²⁴ and R²⁵, together with the carbon atoms to which they are bonded, form a saturated or unsaturated, substituted or unsubstituted 5- to 19-membered carbocyclic ring that optionally contains 1- to 7-heteroatom substitutions selected from O, N, or S as ring member(s), wherein at least one of Ar² and Ar³ is a moiety of formula F2, unless R¹, R², or R⁴ is OH, wherein at least one of R¹, R², or R⁴ is OH when Ar¹ is a moiety of formula C, wherein at least one of R¹, R², R⁴, or R¹¹ is OH when Ar¹ is a moiety of formula D.

3. (canceled)

4. (canceled)

5. (canceled)

6. A composition of a liquid, gel, or emulsion comprising the compound according to claim 2, wherein the compound is dissolved or dispersed at a concentration of 1 wt % or greater in a fluid comprising at least water or an alcohol.

7. The composition of claim 6 further comprising one or more additive(s) of organic solvents, cosmetic oils, silicone fluids, rheological modifiers such as thickeners or thixotropic agents, surfactants or emulsifiers, emollients, humectants, film formers, antimicrobial agents, washable dyes, or combinations thereof.

8. The composition of claim 7, wherein the water phase may further contain saline or phosphate buffer; the alcohols are selected from the group consisting of ethanol, isopropanol, 1,3-propanediol, propylene glycol, 1-propanol, 1,3-butanediol, 1,4-butanediol, glycerol, stearyl alcohol, cetyl alcohol, oleyl alcohol, 1-hexanol, 1-heptanol, 1-decanol, 1,3-dioxolane, 1,6-hexanediol, 2-butoxyethanol, 2-(2-butoxyethanol), 2-(2-ethoxy)ethyl acetate, 2-(2-ethoxyethoxy)ethanol, 2-(2-methoxyethoxy)ethanol 2-ethoxyethanol, 2-methoxyethanol, geraniol, and 1,10-decanediol, the organic solvents are selected from the group consisting of ethyl acetate, isopropyl myristate, isopropyl stearate, coco-caprylate, squalene, n-decane, n-hexadecane, dodecane, lauraldehyde, n-octane, (+/−)-limonene, p-cymene, methyl laurate, ethyl oleate, 2-furancarboxyaldehyde, amyl alcohol, decafluoropentane, acetone, isododecane, 2-butanone, 2-butoxyethyl acetate, 2-ethoxyethyl acetate, amyl acetate, and butyl methyl ketone; the cosmetic oils are selected from the group consisting of açaí oil, agar oil, ajwain oil, algal oil, almond oil, amaranth oil, angelica root oil, apple seed oil, apricot oil, anise oil, argan oil, artichoke oil, astrocaryum murumuru butter, attar, avocado oil, babassu oil, basil oil, bay leaf oil, beech nut oil, ben oil, bergamot oil, birch oil, bitter gourd oil, blackcurrant seed oil, black seed oil, bladderpod oil, borage seed oil, Borneo tallow nut oil, bottle gourd oil, Brazil nut oil, buffalo gourd oil, burdock oil, buriti oil, butternut squash seed oil, calamus oil, camphor oil, candlenut oil, cannabis flower oil, canola oil, cape chestnut oil, caraway seed oil, cardamom seed oil, carob pod oil, carrot seed oil, cashew oil, castor oil, cedar oil, chamomile oil, cinnamon oil, citronella oil, clary sage oil, clove oil, cocklebur oil, cocoa butter, coconut oil, cocoa butter, cohune oil, colza oil, coriander seed oil, corn oil, cottonseed oil, cypress oil, date seed oil, dika oil, egusi seed oil, evening primrose oil, eucalyptus oil, false flax oil, fennel seed oil, flaxseed oil, frankincense oil, grape seed oil, grapefruit seed oil, hazelnut oil, hemp oil, hickory nut oil, Jamaican cobnut oil, jasmine oil, jojoba oil, kapok seed oil, kenaf seed oil, lallemantia oil, lavender oil, lemon oil, lemongrass oil, linseed oil, orange oil, macadamia oil, mafura oil, mango oil, marula oil, meadowfoam seed oil, mongogo nut oil, mowrah butter, mustard seed oil, olive oil, Niger seed oil, nutmeg butter, ojon oil, okra seed oil, palm oil, palo santo oil, papaya seed oil, parsley oil, peanut oil, pecan oil, peppermint oil, perilla seed oil, persimmon seed oil, pequi oil, pili nut oil, pine nut oil, pistachio oil, pomegranate seed oil, poppyseed oil, pracaxi oil, prune kernel oil, pumpkin seed oil, quinoa oil, radish oil, ramtil oil, rapeseed oil, rice bran oil, royle oil, rose oil, rosehip oil, rosewood oil, rubber seed oil, sacha inchi oil, safflower oil, sage oil, salicornia oil, sandalwood oil, sapote oil, sassafras oil, savory oil, seje oil, shea butter, soybean oil, spruce oil, sunflower oil, tall oil, tangerine oil, taramira oil, tea seed oil, tea tree oil, thistle oil, thyme oil, tigernut oil, tobacco seed oil, tomato seed oil, tonka bean oil, walnut oil, watermelon seed oil, wintergreen oil, wheat germ oil, and yarrow oil; the silicone fluids are selected from the group consisting of dimethicone, cyclomethicone, caprylyl methicone, decamethyltetrasiloxane, decamethylcyclopentasiloxane, cycloethoxymethicone, cycloheptasiloxane, cyclohexasiloxane, dimethiconol, phenyl trimethicone, and cyclotrisiloxane; the thickening agents are selected from the group consisting of xanthan gum, gum arabic, carrageenan, tragacanth gum, guar gum, tara gum, locust bean gum, agar agar, konjac gum, gellan gum, polyacrylates, poly(ethylene glycol) (PEG), and PEG derivatives such as PEG stearates; the thixotropic agents are selected from the group consisting of colloidal silica, bentonite, magnesium aluminum silicate, and polymerized vegetable oil; the surfactants are selected from the group consisting of TWEEN 20, TWEEN 40, TWEEN 60, TWEEN 80, polysorbate 20, polysorbate 40, polysorbate 60, polysorbate 80, decyl glucoside, sorbitan monolaurate, sodium lauryl sulfate, cocamidopropyl betaine, glyceryl stearate, PEG-100 stearate, sodium cocoamphoacetate, sodium cocoyl isethionate, sodium methyl cocoyl taurate, disodium laureth sulfosuccinate, and sodium lauroyl sarcosinate; the film-forming agents are selected from the group consisting of polyacrylates, acrylate copolymers, carbomers, polyvinylpyrrolidone, polyvinyl alcohol, polyethers, silicone resins, cellulose and cellulose derivatives such as ethylcellulose or hydroxyethylcellulose, polyurethanes, polyesters, polyquaterniums, polymethyl methacrylate, and natural resins; the emollients are selected from the cosmetic oils, petrolatum, mineral oil, lanolin, C₁₄-C₂₂alkanes, C₉-C₁₆ isoparaffins, caprylic triglycerides, lauric triglycerides, cetyl salicylate, and soluble collagen; the humectants are selected from the group consisting of glycerol, hyaluronic acid, propylene glycol, sorbitol, butylene glycol, urea, alpha hydroxy acids such as lactic acid, glycolic acid, tartaric acid, citric acid, or mandelic acid, sodium pyrrolidone carboxylic acid, sodium hyaluronate, polyethylene glycol (PEG), honey, aloe vera, panthenol, sodium lactate, and betaine; the antiseptics or antimicrobial agents are selected from the group consisting of benzalkonium chloride, benzethonium chloride, chlorhexidine, triclosan, phenoxyethanol, ethylhexylglycerin, benzoyl peroxide, silver compounds such as silver sulfadiazine, iodopropynyl butylcarbamate, and zinc pyrithione; and the washable dyes are selected from the group consisting of FD&C Red No. 40 (Allura Red), FD&C Yellow No. 5 (Tartrazine), FD&C Blue No. 1 (Brilliant Blue), D&C Red No. 27 (Erythrosine), D&C Red No. 7 (Acid Red), D&C Yellow No. 10 (Quinoline Yellow), beetroot extract, turmeric extract, and spirulina extract.

9. The use of a composition according to claim 6 as a topical sunscreen for human or other mammalian skin.

10. The use of a composition according to claim 6 as a topical ink for cosmetic body art on human or other mammalian skin.

11. A skin-marking device containing a composition according to claim 6 impregnated in the reservoir of a pen or marker possessing a ball-point tip, felt or sponge tip, brush tip, or fountain tip.

12. A skin-marking device containing a composition of claim 7 as a thin film on a flexible support layer of paper, plastic, or fabric.

13. A method of applying a composition of claim 6, comprising the steps of: (1) contacting the skin with a marker, brush, pen, sponge or other dispensing media having the composition of claim 6; and (2) marking the skin with the composition.

14. A method of applying a composition of claim 6, comprising the steps of: (1) ejecting droplets of the composition from an air-brush machine, aerosol actuator, bag-on-valve actuator, spray nozzle or other atomizing media; and (2) contacting the skin with atomized composition containing the ortho-phthalaldehyde ultraviolet absorber or visible chromophore.

15. (canceled)

16. A process for the preparation of a compound of formula A according to claim 2, comprising at least the step of reacting at least one compound of formula N, selected from the group consisting of formulae N1, N2, N3, or N4, 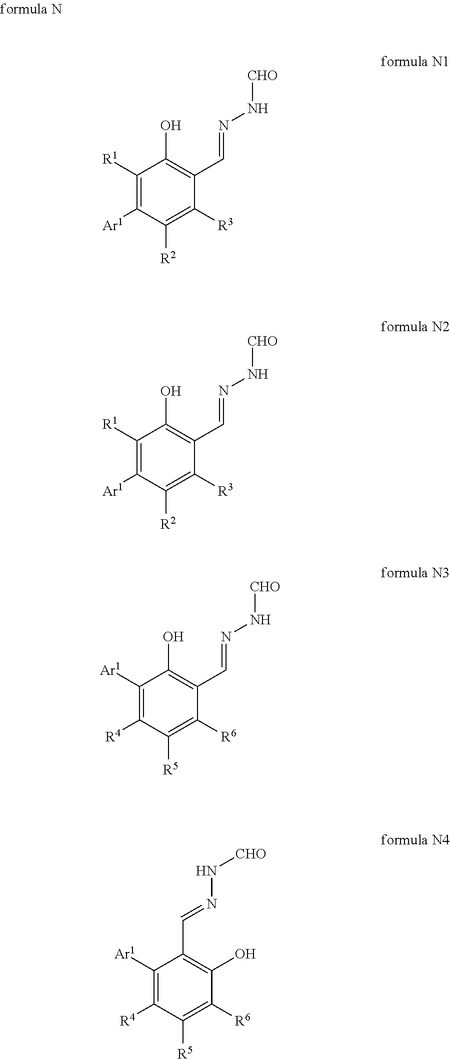
wherein Ar¹ is an ultraviolet absorbing moiety of formula B, formula C, formula D, or formula E, 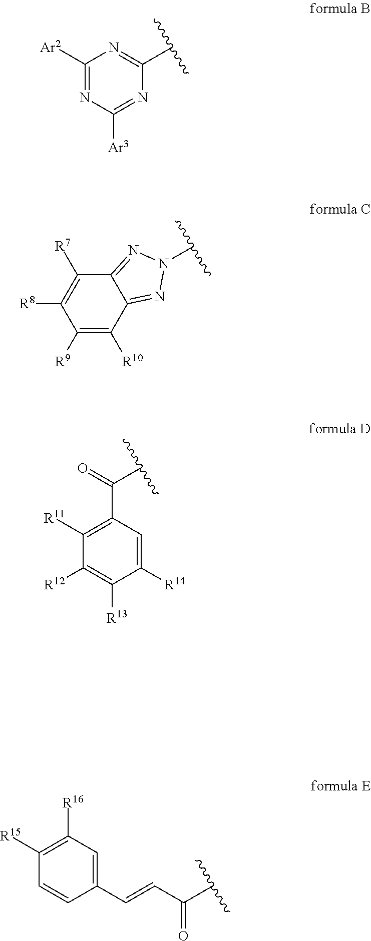
wherein Ar² and Ar³ are independently of each other a moiety of formula F, selected from the group consisting of formulae F1 or F2, 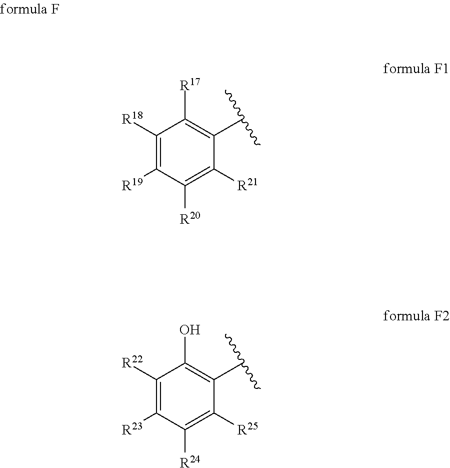
R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, and R²⁵ are independently selected from the group consisting of hydrogen, linear or branched C1-C24 alkyl, linear or branched C2-C24 alkenyl, linear or branched C2-C24 alkynyl, substituted or unsubstituted C₅-C₂₄ cycloalkyl, substituted or unsubstituted C₆-C₂₄ aryl, substituted or unsubstituted C₇-C₂₅ arylalkyl, halogen, CHO, C(═O)R, C(═O)OR, C(═O)OM, CN, OR, NRR′, NO2, SR, S(═O)2R, S(═O)2OR, or S(═O)2OM wherein M is an alkali metal, and R and R′ are independently selected from the group consisting of hydrogen, linear or branched C₁-C₂₄ alkyl, substituted or unsubstituted C₆-C₂₄aryl, substituted or unsubstituted C7-C25 arylalkyl, or R² and R³, together with the carbon atoms to which they are bonded, or R⁴ and R⁵, together with the carbon atoms to which they are bonded, or R⁵ and R⁶, together with the carbon atoms to which they are bonded, or R⁷ and R⁸, together with the carbon atoms to which they are bonded, or R⁸ and R⁹, together with the carbon atoms to which they are bonded, or R⁹ and R¹⁰, together with the carbon atoms to which they are bonded, or R¹¹ and R¹², together with the carbon atoms to which they are bonded, or R¹² and R¹³, together with the carbon atoms to which they are bonded, or R¹³ and R¹⁴, together with the carbon atoms to which they are bonded, or R¹⁵ and R¹⁶, together with the carbon atoms to which they are bonded, or R¹⁷ and R¹⁸, together with the carbon atoms to which they are bonded, or R¹⁸ and R¹⁹, together with the carbon atoms to which they are bonded, or R¹⁹ and R²⁰, together with the carbon atoms to which they are bonded, or R²⁰ and R²¹, together with the carbon atoms to which they are bonded, or R²² and R²³, together with the carbon atoms to which they are bonded, or R²³ and R²⁴, together with the carbon atoms to which they are bonded, or R²⁴ and R²⁵, together with the carbon atoms to which they are bonded, form a saturated or unsaturated, substituted or unsubstituted 5- to 19-membered carbocyclic ring that optionally contains 1- to 7-heteroatom substitutions selected from O, N, or S as ring member(s), and wherein at least one of Ar² and Ar³ is a moiety of formula F2, unless R¹, R², or R⁴ is OH (the hydroxyl group), wherein at least one of R¹, R², or R⁴ is an OH hydroxyl group when Ar¹ is a moiety of formula C, wherein at least one of R¹, R², R⁴, or R¹¹ is an OH hydroxyl group when Ar¹ is a moiety of formula D, with lead tetraacetate, phenyliodine diacetate, or poly(styrene-(iodoso diacetate).

17. A process for the preparation of a compound of formula A according to claim 2, comprising at least the step of reacting at least one compound of formula O, selected from the group consisting of formulae O1, O2, O3, or O4, 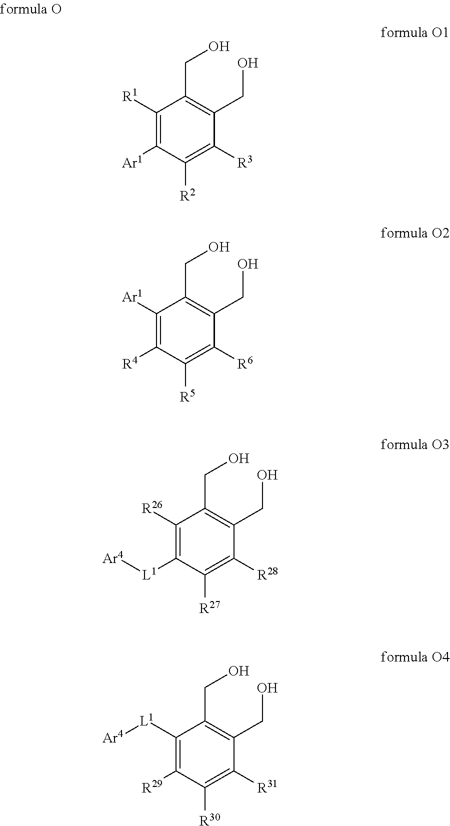
wherein Ar¹ is an ultraviolet absorbing moiety of formula B, formula C, formula D, or formula E, 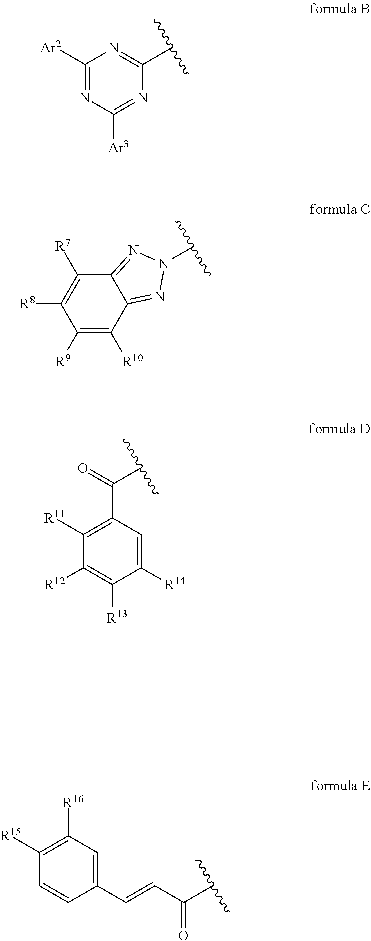
Ar⁴ is a moiety of formula D, or a moiety of formula E, 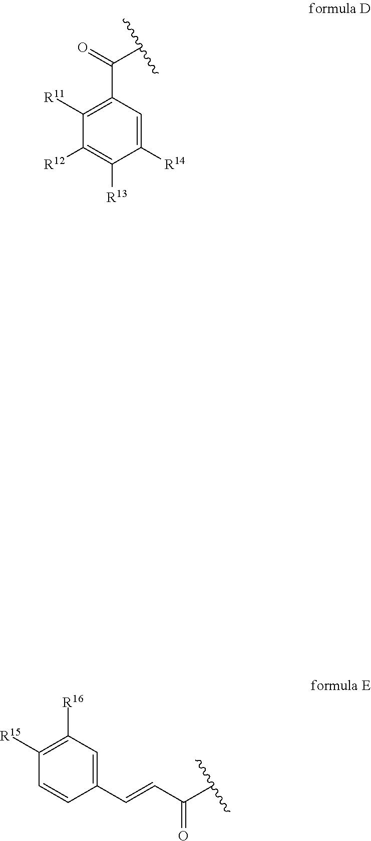
or a moiety of formula H, selected from the group consisting of formulae H1, H2, or H3, 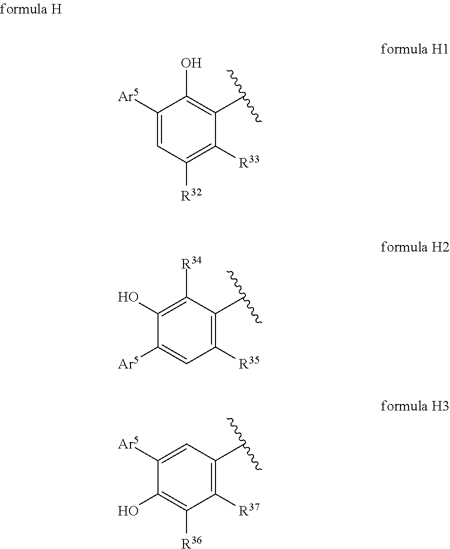
or a moiety of formula I, selected from the group consisting of formulae I1, I2, or I3, 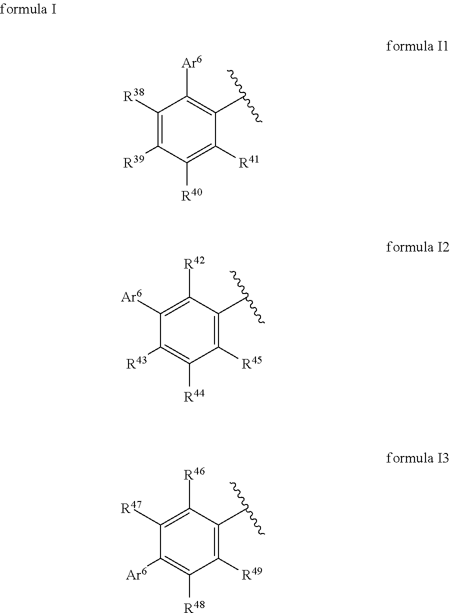
wherein Ar⁵ and Ar⁶ are independently of each other a moiety of formula B, a moiety of formula C, a moiety of formula D, or a moiety of formula E, 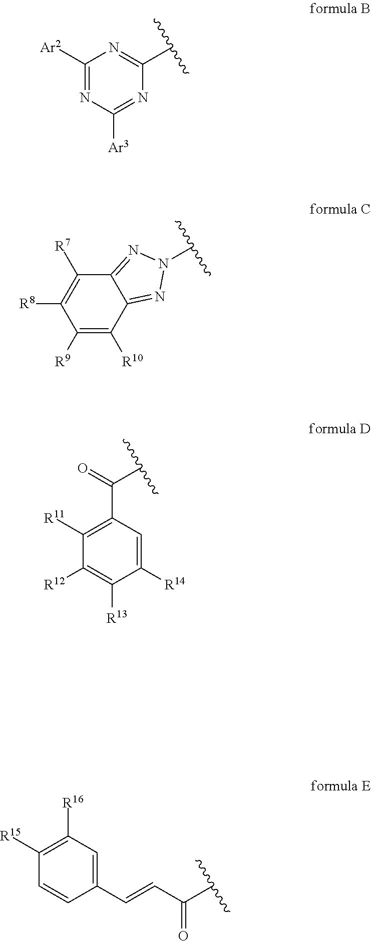
wherein Ar² and Ar³ are independently of each other a moiety of formula F, selected from the group consisting of formulae F1 or F2, wherein R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, and R⁴⁹, are independently of each other selected from the group consisting of hydrogen, linear or branched C₁-C₂₄ alkyl, linear or branched C₂-C₂₄ alkenyl, linear or branched C2-C24 alkynyl, substituted or unsubstituted C5-C24 cycloalkyl, substituted or unsubstituted C6-C24 aryl, substituted or unsubstituted C7-C25 arylalkyl, halogen, C(═O)R, C(═O)OR, C(═O)OM, CN, OR, NRR′, NO₂, SR, S(═O)₂R, S(═O)₂OR, or S(═O)₂OM wherein M is an alkali metal, and R and R′ are independently selected from the group consisting of hydrogen, linear or branched C1-C24 alkyl, substituted or unsubstituted C6-C24 aryl, substituted or unsubstituted C₇-C₂₅ arylalkyl, or R² and R³, together with the carbon atoms to which they are bonded, or R⁴ and R⁵, together with the carbon atoms to which they are bonded, or R⁵ and R⁶, together with the carbon atoms to which they are bonded, or R⁷ and R⁸, together with the carbon atoms to which they are bonded, R⁷ and R⁸, together with the carbon atoms to which they are bonded, or R⁸ and R⁹, together with the carbon atoms to which they are bonded, or R⁹ and R¹⁰, together with the carbon atoms to which they are bonded, or R¹¹ and R¹², together with the carbon atoms to which they are bonded, or R¹² and R¹³, together with the carbon atoms to which they are bonded, or R¹³ and R¹⁴, together with the carbon atoms to which they are bonded, or R¹⁵ and R¹⁶, together with the carbon atoms to which they are bonded, or R¹⁷ and R¹⁸, together with the carbon atoms to which they are bonded, or R¹⁸ and R¹⁹, together with the carbon atoms to which they are bonded, or R¹⁹ and R²⁰, together with the carbon atoms to which they are bonded, or R²⁰ and R²¹, together with the carbon atoms to which they are bonded, or R²² and R²³, together with the carbon atoms to which they are bonded, or R²³ and R²⁴, together with the carbon atoms to which they are bonded, or R²⁴ and R²⁵, together with the carbon atoms to which they are bonded, or R²⁷ and R²⁸, or R²⁹ and R³⁰, together with the carbon atoms to which they are bonded, or R³⁰ and R³¹, together with the carbon atoms to which they are bonded, or R³² and R³³, together with the carbon atoms to which they are bonded, or R³⁶ and R³⁷, together with the carbon atoms to which they are bonded, or R³⁸ and R³⁹, together with the carbon atoms to which they are bonded, or R³⁹ and R⁴⁰, together with the carbon atoms to which they are bonded, or R⁴⁰ and R⁴¹, together with the carbon atoms to which they are bonded, or R⁴³ and R⁴⁴, together with the carbon atoms to which they are bonded, or R⁴⁴ and R⁴⁵, together with the carbon atoms to which they are bonded, or R⁴⁶ and R⁴⁷, together with the carbon atoms to which they are bonded, or R⁴⁸ and R⁴⁹, together with the carbon atoms to which they are bonded, form a saturated or unsaturated, substituted or unsubstituted 5- to 19-membered carbocyclic ring that optionally contains 1- to 7-heteroatom substitutions selected from O, N, or S as ring member(s), L¹ is an optional substituted or unsubstituted linking unit comprising a moiety of formula J, selected from the group consisting of formulae J1, J2, J3, J4, J5, J6, J7, J8, J9, J10, J11, J12, J13, J14, J15, or J16, 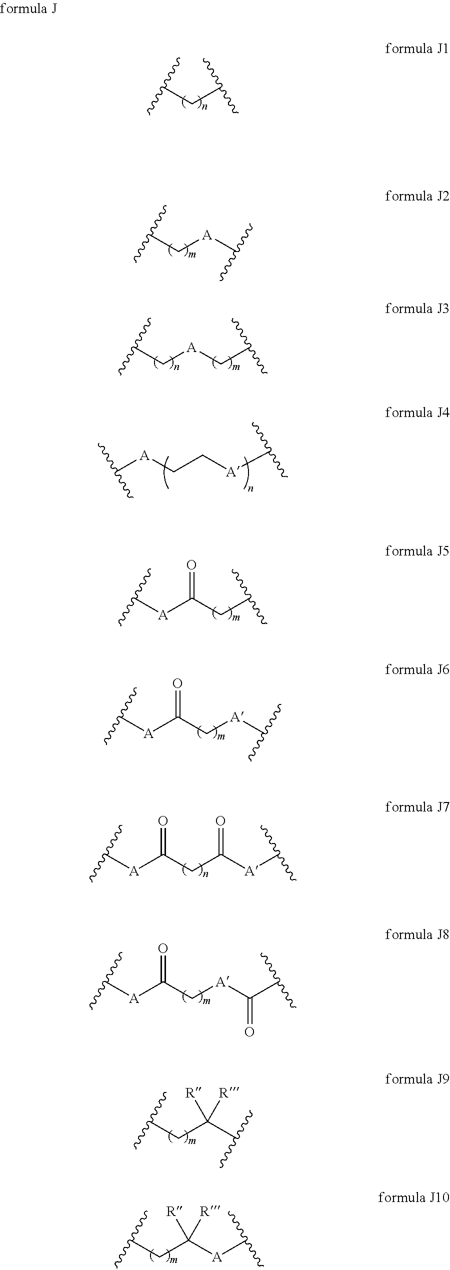 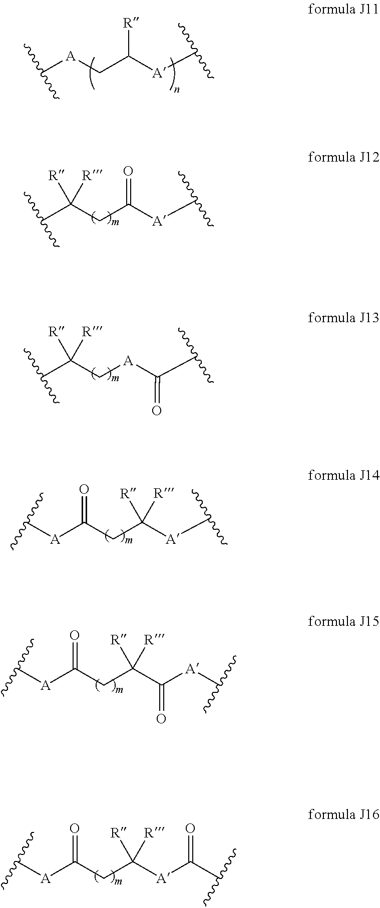
or an optional 1,2-, 1,3-, or 1,4-substituted phenylene linking unit comprising a moiety of formula K, selected from the group consisting of formulae K1, K2, K3, K4, K5, K6, K7, K8, K9, K10, K11, K12, K13, K14, or K15, 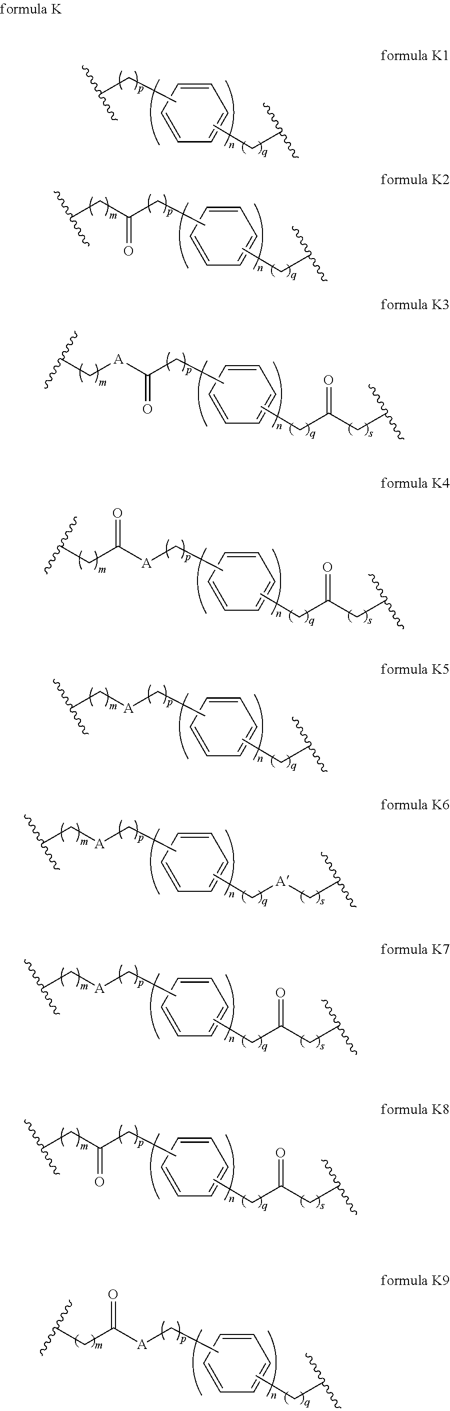 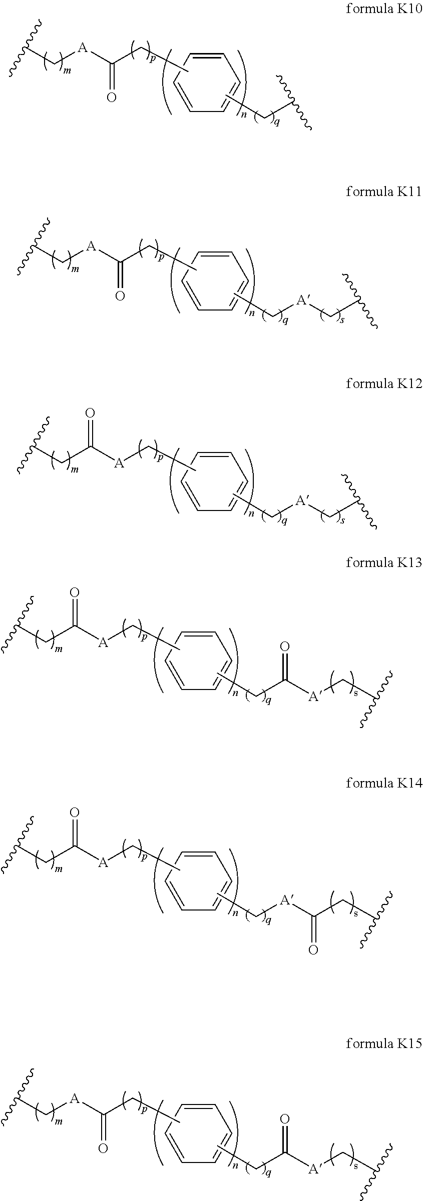
wherein A and A′ are, independently of each other, linking units selected from the group consisting of O, NH, S, SO2, O(C═O), NH(C═O), NH(C═S), or S(C═O), R″ and R′″ are independently of each other selected from the group consisting of hydrogen, linear or branched C₁-C₂₄ alkyl, substituted or unsubstituted C₆-C₂₄aryl, substituted or unsubstituted C₇-C₂₅ arylalkyl, C(═O)R, C(═O)OR, or OR, wherein R is selected from the group consisting of hydrogen, linear or branched C1-C24 alkyl, substituted or unsubstituted C6-C24 aryl, substituted or unsubstituted C₇-C₂₅ arylalkyl, n denotes a repeating unit that may number from 1 to 24, m, p, q, and s independently of each other denote repeating units that may number from 0 to 24, which may be either monodisperse or polydisperse, and wherein the O(C═O), NH(C═O), NH(C═S), or S(C═O) moieties of A and A′, or the unsymmetrical linking units of formula J or formula K may be oriented in either of the two possible directions, and at least one of Ar² and Ar³ is a moiety of formula F2, unless R¹, R², or R⁴ is OH, at least one of R¹, R², or R⁴ is OH when Ar¹ is a moiety of formula C, at least one of R¹, R², R⁴, or R¹¹ is OH when Ar¹ is a moiety of formula D, at least one of Ar² and Ar³ is a moiety of formula F2 when Ar⁴ is a moiety of formula I, unless R³⁸, R⁴², R⁴³, R⁴⁷, or R⁴⁸ is OH, at least one of R³⁸, R⁴², R⁴³, R⁴⁷, and R⁴⁸ is OH when Ar⁶ is a moiety of formula C, and at least one of R¹¹, R³⁸, R⁴², R⁴³, R⁴⁷, and R⁴⁸ is OH when Ar⁶ is a moiety of formula D, with any reagent suitable for oxidizing the ortho-benzylic alcohol moieties of compounds of formula O to the corresponding ortho-benzaldehydes of formula A or formula G, including but not limited to pyridinium chlorochromate, or pyridinium chromate, or oxalyl chloride, or dimethyl sulfoxide with 1-ethyl-3-(3-dimethylaminopropyl) carbodiimide, or dimethyl sulfoxide with diisopropylcarbodiimide, or dimethyl sulfoxide with dicyclohexylcarbodiimide, or dimethyl sulfoxide with acetic anhydride, or dimethyl sulfoxide with phosphorus pentoxide, or dimethyl sulfoxide with sulfur trioxide, or Dess-Martin periodinane, or ortho-iodoxybenzoic acid in dimethyl sulfoxide, or manganese oxide, or 2,3-dichloro-5,6-dicyano-p-quinone, or catalytic tetra-n-propylammonium perruthenate, or NaOCl with (2,2,6,6,-tetramethylpiperidin-1-yl)oxanyl.

18. (canceled)

19. (canceled)

20. (canceled)

21. (canceled)

22. (canceled)
